(19) United States
(12) Patent Application Publication
(10) Pub. No.: US 2026/0263651 A1
(43) Pub. Date: Sep. 10, 2026

(54) WOUND DRESSING

(71) Applicant: UNIVERSITY OF SUNDERLAND, Sunderland Tyne and Wear (GB)

(72) Inventors: Mark Gray, Sunderland Tyne and Wear (GB); Anna Adam, Sunderland Tyne and Wear (GB); Giovanna Li Petri, Sunderland Tyne and Wear (GB); Callum J. Cooper, Sunderland Tyne and Wear (GB)

(73) Assignee: UNIVERSITY OF SUNDERLAND, Sunderland Tyne and Wear (GB)

(21) Appl. No.: 19/165,262

(22) PCT Filed: Mar. 15, 2024
(86) PCT No.: PCT/GB2024/050725

§ 371 Date: Sep. 15, 2025

(30) Foreign Application Priority Data
Mar. 16, 2023 (GB) 2303824.3

(51) Int. Cl. A61L 15/28

(52) U.S. Cl. CPC A61L 15/28; A61L 2300/442

(57) ABSTRACT

This invention relates to a wound dressing comprising a modified polymer, wherein the modified polymer comprises a polymer backbone covalently coupled to a compound of Formula (I): (I). X is a covalent bond or a linker; R¹ is independently at each occurrence selected from halo, C₁₋₃ alkyl, C₂₋₃ alkenyl, C₁₋₃ haloalkyl, —O—C₁₋₃ alkyl, —O—C₁₋₃ haloalkyl and CN; and n is 0, 1, 2 or 3. Also provided are a methods of making the wound dressing and uses of such a wound dressing. The invention also relates to a method for detecting the presence of bacteria in a wound comprising applying the aforementioned wound dressing to the wound and a method of forming a modified polysaccharide, said polysaccharide allowing for the detection of bacteria in a wound when part of a wound dressing.

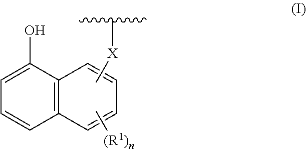

This invention relates to a wound dressing comprising a modified polymer useful for detecting the presence of bacteria in a wound. Also provided are methods of making the wound dressing and uses of such a wound dressing. The invention also relates to a method for detecting the presence of bacteria in a wound comprising applying the aforementioned wound dressing to the wound and a method of forming a modified polysaccharide, said polysaccharide allowing for the detection of bacteria in a wound when part of a wound dressing.

### BACKGROUND

Chronic wounds are defined as those that do not heal via the usual orderly set of stages, often persisting for eight weeks without displaying the standard signs of healing (What Are the Treatment Options for Chronic Wounds?; Institute for Quality and Efficiency in Health Care (IQWiG), 2018). Indeed, those suffering from chronic wounds may suffer them for many months. The risk of developing a chronic wound increases with age (Yao, Z.; Niu, J.; Cheng, B. Prevalence of Chronic Skin Wounds and Their Risk Factors in an Inpatient Hospital Setting in Northern China. Advances in Skin & Wound Care 2020, 33 (9), 1-10) and health conditions, such as diabetes (Greenhalgh, D. G. Wound Healing and Diabetes Mellitus. Clin Plast Surg 2003, 30 (1), 37-45) or cancer (Sundaram, G. M.; Quah, S.; Sampath, P. Cancer: The Dark Side of Wound Healing. The FEBS Journal 2018, 285 (24), 4516-4534). While the treatment of chronic wounds is more complex and expensive than a normal wound, the treatment can be further complicated by the establishment of bacterial infections delaying the healing time and increasing the risk of the pathogenic strains developing antimicrobial resistance. Antimicrobial resistance occurs when certain drugs which were previously inhibitory become ineffective against a microorganism.

The progress of a wound infection can be divided into three stages: contamination, when a nonreplicating microbe is present in the open wound; colonization, when the strains are replicating; and infection, when the increased number of pathogens affect the wound healing process causing inflammation and potential tissue damage when the invasive cells migrate deeper into the wound (Haesler, E.; Ousey, K. Evolution of the Wound Infection Continuum. Wounds International 2018, 9 (4), 6-10; Li, S.; Renick, P.; Senkowsky, J.; Nair, A.; Tang, L. Diagnostics for Wound Infections. Adv Wound Care (New Rochelle) 2021, 10 (6), 317-32). Most wound infections are caused by bacteria, and these are most commonly due to a Staphylococcus aureus, Escherichia coli, Enterococcus spp., or Pseudomonas aeruginosa infection (Bowler, P. G.; Duerden, B. I.; Armstrong, D. G. Wound Microbiology and Associated Approaches to Wound Management. Clin Microbiol Rev 2001, 14 (2), 244-269; Saeed, M. A.; Haque, A.; Ali, A.; Mohsin, M.; Bashir, S.; Tariq, A.; Afzal, A.; Iftikhar, T.; Sarwar, Y. A Profile of Drug Resistance Genes and Integrons in E. Coli Causing Surgical Wound Infections in the Faisalabad Region of Pakistan. J Antibiot 2009, 62 (6), 319-323).

The diagnosis of wound infections is usually based on clinical characteristics or patient-reported symptoms, however wound swab cultures are becoming more frequently taken as they are reliable and inexpensive as a means for pathogen identification (Hsu, J.-T.; Chen, Y.-W.; Ho, T.-W.; Tai, H.-C.; Wu, J.-M.; Sun, H.-Y.; Hung, C.-S.; Zeng, Y.-C.; Kuo, S.-Y.; Lai, F. Chronic Wound Assessment and Infection Detection Method. BMC Med Inform Decis Mak 2019, 19, 99). However, ensuring that samples are free of outside contamination, to minimise false positive results, requires time and skill, which adds to the workload when treating a wound. Moreover, the need to transport the samples to a specialist laboratory coupled with the need to culture the sample over several days means that there is a significant delay before obtaining a result. This means that any infection has an opportunity to significantly progress, making treatment even more difficult. Indeed, it is sadly the case that in some cases chronic wounds become so difficult to treat with antibiotics that debridement of the tissues, or even amputation of the infected limb may become necessary to protect the life of the patient.

Thus, there is a need for a timely diagnostic system, that can be used to determine whether bacterial colonisation has occurred in the point-of-care setting.

Certain enzymatic substrates can provide chromogenic or fluorogenic response in the presence of bacteria via enzymatic hydrolysis. They can be specific for only a narrower range of bacteria (such as substrates for the L-alanyl aminopeptidase, which would be cleaved by Gram-negative strains). However some enzymatic targets (such as the L-Leucyl aminopeptidase) can be considered as common to all bacteria and provide information of a general bacterial presence (Colloms, S. D. Chapter 333—Leucyl Aminopeptidase PepA. In Handbook of Proteolytic Enzymes (Third Edition); Rawlings, N. D., Salvesen, G., Eds.; Academic Press, 2013; pp 1484-1492). M. Cellier, et al., Bioorg. Med. Chem., 22, 2014, 5249-5269 discloses a series of chromogenic aminopeptidase substrates for the detection and identification of clinically important microorganisms.

It is an aim of certain embodiments of the invention to provide a convenient system that allows the presence of bacteria in a wound to be detected, e.g. by providing a colour change to a wound dressing. Upon removing the dressing from the patient, this would allow an observer to see a distinctive colour on the surface of the dressing.

### BRIEF SUMMARY OF THE DISCLOSURE

In accordance with a first aspect of the invention there is provided a wound dressing comprising a modified polymer, wherein the modified polymer comprises a polymer backbone covalently coupled to a compound of Formula (I):

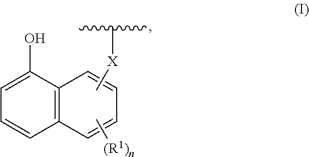

wherein X is a covalent bond or a linker;
R¹ is independently at each occurrence selected from halo, C₁₋₃ alkyl, C₂₋₃ alkenyl, C₁₋₃ haloalkyl, —O—C₁₋₃ alkyl, —O—C₁₋₃ haloalkyl and CN;
n is 0, 1, 2 or 3; and
[symbol] indicates the point of attachment to the polymer backbone.

Covalently coupling the compound of Formula (I) to the polymer backbone provides a single component modified polymer that may be contacted with the surface of a wound.

In accordance with a second aspect of the invention there is provided a sterile package comprising a wound dressing according to the first aspect of the invention. Advantageously, providing the wound dressing in a sterile package minimises the risk that the wound dressing would comprise bacteria before it is applied to a wound.

In accordance with a third aspect of the invention there is provided a use of a wound dressing according to the first aspect of the invention for detecting the presence of bacteria.

In accordance with a fourth aspect of the invention there is provided a method for detecting the presence of bacteria in a wound, the method comprising applying a wound dressing according to the first aspect of the invention to a wound, removing the wound dressing from the wound and observing any colouration on the wound dressing.

In accordance with a fifth aspect of the invention there is provided a method of forming a modified polysaccharide comprising a modified monosaccharide residue of Formula (III):

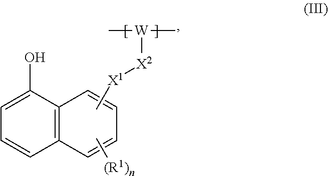

wherein X¹ is a covalent bond or a linker;
X² is selected from NH, O and S;
W is a monosaccharide residue;
R¹ is independently at each occurrence selected from halo, C₁₋₃ alkyl, C₂₋₃ alkenyl, C₁₋₃ haloalkyl, —O—C₁₋₃ alkyl, —O—C₁₋₃ haloalkyl and CN; and
n is 0, 1, 2 or 3,
the method comprising:
a) contacting an acidic polysaccharide with a compound of Formula (IV):

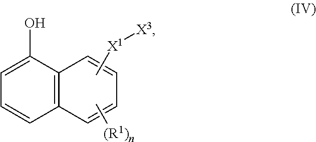

to form the modified polysaccharide,
wherein X³ is selected from —NH₂, —OH and —SH,
with the proviso that when X² is NH, X³ is —NH₂; when X² is O, X³ is —OH; and when X² is S, X³ is —SH.

A sixth aspect provides a modified polysaccharide obtainable by or obtained by the method of the fifth aspect.

In accordance with a seventh aspect of the invention there is provided a method of forming a wound dressing, wherein the wound dressing comprises a modified polysaccharide, wherein the method comprises forming the modified polysaccharide according to the method of the fifth aspect.

An eighth aspect of the invention provides a wound dressing obtainable by or obtained by the method of the seventh aspect.

In accordance with a ninth aspect of the invention there is provided a method of forming a packaged wound dressing, the method comprising:
i) forming a wound dressing according to the method of the seventh aspect;
ii) packaging the wound dressing to form the packaged wound dressing; and
iii) optionally sterilising the packaged wound dressing.

A tenth aspect of the invention provides an (optionally sterile) packaged wound dressing obtainable by or obtained by the method of the ninth aspect.

### BRIEF DESCRIPTION OF THE DRAWINGS

Embodiments of the invention are further described hereinafter with reference to the accompanying drawings, in which:

FIG. 1 shows alginate wound dressing materials modified with: no modification (left), compound 10 (middle left); or compound 8 (middle right and right).

FIG. 2 shows modified alginate wound dressing materials treated with 3a against EC, after overnight incubation. Left plate: upper view; Right plate: bottom view.

FIG. 3 shows modified alginate wound dressing materials treated with 3b against EC, after overnight incubation. Left plate: upper view; Right plate: bottom view.

FIG. 4 shows modified alginate wound dressing materials treated with 3a against PSA, after overnight incubation. Left plate: upper view; Right plate: bottom view.

FIG. 5 shows modified alginate wound dressing materials treated with 3b against PSA, after overnight incubation. Left plate: upper view; Right plate: bottom view.

FIG. 6 shows modified alginate wound dressing materials treated with 3a against SA, after overnight incubation. Left plate: upper view; Right plate: bottom view.

FIG. 7 shows modified alginate wound dressing materials treated with 3b against SA, after overnight incubation. Left plate: upper view; Right plate: bottom view.

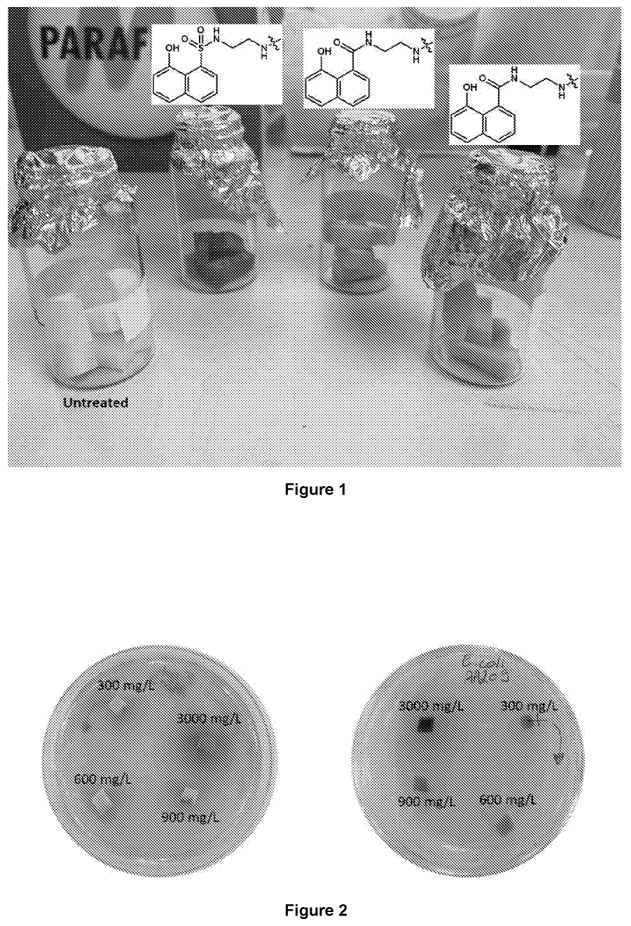

### DETAILED DESCRIPTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Definitions

The term “linker” is intended to cover any organic bivalent radical. Examples of such organic bivalent radicals include alkylene (e.g. C₁-C₁₂ alkylene), a PEG linker (e.g. —(OCH₂CH₂)ₘ— or —(CH₂CH₂O)ₘ—, where m is an integer from 1 to about 100); and/or comprise at least one of —S—, —SO₂— or 1,2,3-triazolylene.

The term “polymer backbone” is intended to cover the (unmodified) polymer from which the modified polymer derives. In other words, the “polymer backbone” is the polymer that is covalently coupled to the compound of Formula (I) in order to form the modified polymer. Suitable polymer backbones for a wound dressing may comprise a synthetic polymer, such as polyurethane, or a polysaccharide, such as cellulose, carboxymethylcellulose, carboxymethyl starch, pectin, alginic acid, carrageenan, heparin, gellan gum, agarose, hyaluronic acid, chitosan or a pharmaceutically acceptable salt thereof.

A biopolymer is any natural polymer formed by living organisms, such as proteins, polynucleotides and polysaccharides.

Polysaccharides are polymeric chains (often with branching) comprising multiple monosaccharide units bound together by glycosidic bonds. Naturally occurring polysaccharides may be found in, e.g. plants, animals, microbes and algae. The term “polysaccharide” is however also intended to include synthetic or synthetically modified polysaccharides, e.g. polysaccharides that have been sulphated, phosphorylated or carboxymethylated.

Acidic polysaccharides are polysaccharides containing an acidic group, often—COOH (or —COO⁻) and/or —OSO₃H (or —OSO₃⁻). The acidic group may be in the free form (i.e. —COOH and —OSO₃H) or in the form of a salt, i.e. wherein the —COO⁻ and/or —OSO₃⁻ group is paired with a suitable counterion, e.g. sodium, potassium, calcium, or ammonium. The acidic group may also be naturally esterified, e.g. with a methoxy or ethoxy group.

The “monosaccharide residue” (W) is the monosaccharide unit of the acidic polysaccharide that is modified thereby forming the modified monosaccharide residue of Formula (III). For example, if W is guluronic acid, then the modified monosaccharide residue of Formula (III) may have the structure:

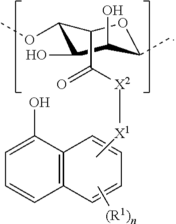

The “amino acid” (that is bonded to the rest of the enzymatic substrate via an amide bond) will typically be a proteinogenic amino acid, i.e. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, selenocysteine or pyrrolysine. The amino acid may be the α-amino acid, e.g. α-leucine, or the β-amino acid, e.g. β-alanine. Typically, the amino acid will be the L-amino acid.

The term “pendant group” (or side group) is an offshoot, neither oligomeric nor polymeric, from a chain. For example, the phenyl groups are the pendant groups on a polystyrene chain.

A “semi-occlusive dressing” (semi-permeable dressing) allows the wound to “breathe” (air can penetrate in and out) whilst protecting the wound from outside liquids. Occlusive dressings are both air- and water-tight.

The wound dressing of the present invention allows the presence of bacteria in a wound to be indicated by coloration (a colour change) on the surface of the wound dressing. The presence of a chromophore causes coloration. The chromophore may be a blue chromophore that causes blue coloration (blue light typically having a wavelength of between 450 and 495 nm). A blue chromophore will therefore typically absorb light having a wavelength outside of the range 450 to 495 nm.

The term Cₘ₋ₙ refers to a group with m to n carbon atoms.

The term “alkyl” refers to a monovalent linear or branched saturated hydrocarbon chain. For example, C₁₋₃ alkyl may refer to methyl, ethyl, n-propyl or iso-propyl.

The term “alkylene” refers to a divalent linear or branched saturated hydrocarbon chain. For example, C₁₋₈ alkylene may refer to methylene, ethylene, n-propylene, iso-propylene, n-butylene, sec-butylene, tert-butylene, n-pentylene, n-hexylene or n-heptylene. The term “alkylene” is synonymous with the term “alkanediyl”.

The term “alkenyl” refers to refers to a monovalent linear or branched saturated hydrocarbon chain containing at least one double bond. The double bond(s) may be present as the E or Z isomer. The double bond may be at any possible position of the hydrocarbon chain. For example, “C₂₋₃ alkenyl” may refer to ethenyl or propenyl.

The term “alkenylene” refers to a bivalent linear or branched saturated hydrocarbon chain containing at least one double bond. The double bond(s) may be present as the E or Z isomer. The double bond may be at any possible position of the hydrocarbon chain. For example, “C₂₋₈ alkenylene” may refer to ethenylene, propenylene, butenylene, butadienylene, pentenylene, pentadienylene, hexenylene, hexadienylene, heptenylene, heptadienylene or octenylene.

The term “alkynyl” refers to refers to a monovalent linear or branched saturated hydrocarbon chain containing at least one triple bond. The triple bond may be at any possible position of the hydrocarbon chain. For example, “C₂₋₄ alkynyl” may refer to ethynyl, propynyl or butynyl.

The term “alkynylene” refers to refers to a bivalent linear or branched saturated hydrocarbon chain containing at least one triple bond. The triple bond may be at any possible position of the hydrocarbon chain. For example, “C₂₋₈ alkynyl” may refer to ethynylene, propynylene, butynylene, pentynylene, hexynylene, heptynylene or octynylene.

The term “haloalkyl” refers to an alkyl group (alkyl being defined as above) where one or more hydrogen atoms are substituted by a corresponding number of halogens. For example, the term “C₁₋₃ haloalkyl” is intended to cover any saturated straight or branched chain alkyl moiety having from 1 to 3 carbon atoms where one or more hydrogen atoms are substituted by a corresponding number of halogen atoms. For example, “C₁₋₃ haloalkyl” covers, but is not limited to, trifluoromethyl (—CF₃), 2,2,2-trifluoroethyl, 3-bromopropyl, and the like.

The term “arylene” refers to any bivalent aromatic carbocyclic ring system (i.e. a ring system containing 2(2n+1)π electrons). The arylene may be monocyclic or polycyclic. The arylene group may have from 6 to 12 carbon atoms in the ring system. Arylene groups will typically be phenylene groups. Arylene groups may be also be naphthylene groups or biphenylene groups.

The term “heteroarylene” refers to any bivalent aromatic (i.e. a ring system containing 2(2n+1)π electrons) 5 to 12 membered ring system comprising from 1 to 4 heteroatoms independently selected from O, S and N (in other words from 1 to 4 of the atoms forming the ring system are selected from O, S and N, the rest being carbon). The heteroarylene may be monocyclic or and polycyclic. The heteroarylene may be a 6-membered heteroarylene in which the heteroaromatic ring is substituted with 1-3 (e.g. 1-2) nitrogen atoms, e.g. pyridinylene.

The term “substituted” as used herein in reference to a moiety means that one or more, e.g. 1, 2 or 3, of the hydrogen atoms in said moiety are replaced independently of each other by the corresponding number of the described substituents.

It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognised by the skilled person.

Where steric issues determine placement of substituents on a group, the isomer having the lowest conformational energy may be preferred.

Where a compound, moiety, process or product is described as “optionally” having a feature, the disclosure includes such a compound, moiety, process or product having that feature and also such a compound, moiety, process or product not having that feature. Thus, when a moiety is described as “optionally substituted”, the disclosure comprises the unsubstituted moiety and the substituted moiety.

Where two or more moieties are described as being “independently” or “each independently” selected from a list of atoms or groups, this means that the moieties may be the same or different. The identity of each moiety is therefore independent of the identities of the one or more other moieties.

The term “pharmaceutically acceptable” as used herein includes reference to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio. This term includes acceptability for both human and veterinary purposes.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centres) or double bonds; the racemates, diastereomers, tautomers, geometric isomers and individual isomers are encompassed within the scope of the present invention. The compounds of the present invention do not include those which are known in the art to be too unstable to synthesize and/or isolate.

The present invention also includes all pharmaceutically acceptable isotopically-labelled compounds of formulae (I) to (V) (as well as isotopically labelled structural units of formulae (A), (Aa), (Ai) to (Aii), and (Bi) to (Biii)), wherein one or more atoms are substituted by an isotope(s) of the same element.

Examples of stable isotopes suitable for inclusion in the compounds of the invention include isotopes of hydrogen, such as ²H, carbon, such as ¹¹C and ¹³C, nitrogen, such as ¹⁵N and oxygen, such as ¹⁷O and ¹⁸O.

Isotopically-labelled compounds can generally be prepared by conventional techniques known to those skilled in the art or by processes analogous to those described using an appropriate isotopically-labelled reagent in place of the non-labelled reagent previously employed.

Isotopic abundance can be determined using conventional analytical methods known to a person skilled in the art, such as mass spectrometry and nuclear magnetic resonance spectroscopy.

Throughout this specification these abbreviations have the following meanings:

| | |
|---|---|
| cc. | concentrated |
| CFU | colony-forming unit |
| Da | Dalton |
| DCC | N,N′-Dicyclohexylcarbodiimide |
| DCM | dichloromethane |
| DI | deionised (water) |
| DIC | N,N′-Diisopropylcarbodiimide |
| DMF | dimethylformamide |
| DMSO | dimethyl sulfoxide |
| EC | Escherichia coli |
| EDC | 1-Ethyl-3-(3- dimethylaminopropyl)carbodiimide |
| ESI | electrospray ionization |
| FT-IR | fourier-transform infrared spectroscopy |
| HPLC | high-performance liquid chromatography |
| HRMS | high-resolution mass spectrometry |
| IBCF | isobutyl chloroformate |
| IR | infrared |
| LRMS | low resolution mass spectrometry |
| LB | lysogeny broth |
| M | molar concentration |
| m.p. | melting point |
| NMM | N-Methylmorpholine |
| OD | optical density |
| PBS | phosphate buffered saline |
| PSA | Pseudomonas aeruginosa |
| r.t. | room temperature |
| SA | Staphylococcus aureus |
| TEMPO | 2,2,6,6-Tetramethylpiperidine 1-oxyl |
| THF | tetrahydrofuran |
| TLC | thin-layer chromatography |
| TSA | tryptic soy agar |

### Wound Dressing

A first aspect of the invention provides a wound dressing comprising a modified polymer, wherein the modified polymer comprises a polymer backbone covalently coupled to a compound of Formula (I):

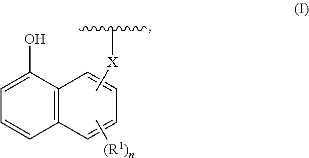

wherein X is a covalent bond or a linker;
R¹ is independently at each occurrence selected from halo, C₁₋₃ alkyl, C₂₋₃ alkenyl, C₁₋₃ haloalkyl, —O—C₁₋₃ alkyl, —O—C₁₋₃ haloalkyl and CN;
n is 0, 1, 2 or 3; and
[symbol] indicates the point of attachment to the polymer backbone.

### Formula (I)

It may be that X is a covalent bond. Typically, however, X is a linker. It may be that the linker has a molecular weight of less than 500 Da. It may be that the linker has a molecular weight of less than 250 Da. It may be that the linker has a molecular weight of less than 150 Da. It may be that the linker has a molecular weight of less than 100 Da.

The linker may comprise a C₁₋₈ alkylene, a PEG linker (i.e. —(OCH₂CH₂)ₘ— or —(CH₂CH₂O)ₘ—), —S—, —SO₂— or 1,2,3-triazolylene, wherein m is an integer selected from 1 to 20, and C₁₋₈ alkylene is optionally substituted with from 1 to 4 substituents selected from oxo, halo, —O—C₁₋₃alkyl, —O—C₁₋₃ haloalkyl and CN.

It may be that the modified polymer is the product of a click reaction. Click reactions are well known in the art. Click reactions include thiol-ene reactions (see, e.g. A. B. Lowe, Polym. Chem., 2020, 1,17-36), thiol-yne reactions (see, e.g. R. Hoogenboom, Angew. Chem. Int. Ed., 2010, 49, 3415-7), SuFEx (see, e.g. J. Dong, L. Krasnova, M. G. Finn, K. B. Sharpless,. Angew. Chem. Int. Ed., 2014, 53, 9430-9448) and azide-alkyne cycloaddition (see, e.g. F. Himo, T. Lovell, R. Hilgraf, V. V. Rostovtsev, L. Noodleman, K. B. Sharpless, V. V. Fokin, J. Am. Chem. Soc., 2005, 127, 210-216). If the modified polymer is synthesised via a thiol-ene reaction, it may be that the linker comprises —S—, e.g. —S—CH₂CH₂—. If the modified polymer is synthesised via a thiol-yne reaction, it may be that the linker comprises —S—, e.g. —S—CH═CH—. If the modified polymer is synthesised via SuFEx, it may be that the linker comprises —SO₂—. If the modified polymer is synthesised via azide-alkyne cycloaddition, it may be that the linker comprises 1,2,3-triazolylene, e.g.

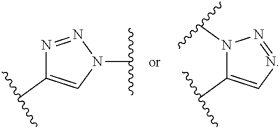 [SMILES: *c1cn(*)nn1 ; *c1cnnn1*]

It may be that the linker comprises —Y—Z¹-L¹-Z²—, wherein Y is selected from —C(O)—, —S(O)₂— and —S(O)—; Z¹ and Z² are independently selected from O, S and NH; and L¹ is selected from C₁₋₈ alkylene, C₂₋₈ alkenylene and C₂₋₈ alkynylene, wherein L¹ is optionally substituted where chemically possible with from 1 to 4 substituents selected from oxo, halo, —O—C₁₋₃ alkyl, —O—C₁₋₃ haloalkyl and CN. It may be that the linker is —Y—Z¹-L¹-Z².

It may be that Y is selected from —C(O)— and —S(O)₂—. It may be that Y is —C(O)—. It maybe that Z¹ is NH. It maybe that Z² is NH. It may be that Z¹ and Z² are each NH. It may be that L¹ is C₁₋₈ alkylene, e.g. C₁₋₄ alkylene. L¹ may be unsubstituted.

It may be that R¹ is independently at each occurrence selected from halo and C₁₋₃ alkyl. It may be that n is 0, 1 or 2. It may be that n is 0 or 1. Preferably, n is 0.

It may be that the compound of Formula (I) is a compound of Formula (Ia):

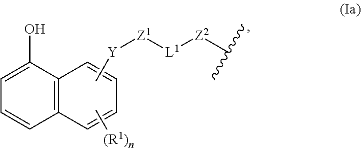

wherein Y, Z¹, Z², L¹, R¹ and n are as defined above.

It may be that the compound of Formula (I) is a compound of Formula (Ib):

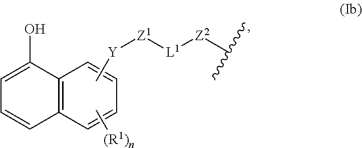

wherein Y, Z¹, Z², L¹, R¹ and n are as defined above.

It may be that the compound of Formula (I) is selected from:

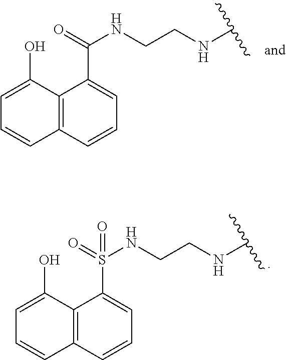 [SMILES: *NCCNC(=O)c1cccc2cccc(O)c12 ; *NCCNS(=O)(=O)c1cccc2cccc(O)c12]

### Polymer Backbone

Typically, the polymer backbone will comprise a pendant group. This pendant group will be reactive and will allow for (or, will have allowed for) the covalent coupling to the compound of Formula (I). It may be that the pendant group is, or comprises, a group selected from oxo, ═NR^{a}, ═NOR^{a}, halo, —NO₂, —CN, —N₃, —NR^{a}R^{a}, —NR^{a}S(O)₂R^{a}, —NR^{a}C(O)R^{a}, —NR^{a}CONR^{a}R^{a}, —NR^{a}CO₂R^{a}, —OR^{a}, —SR^{a}, —S(O)R^{a}, —S(O)₂R^{a}, —S(O)₂F, —SO₃H, —OSO₃H, —S(O)₂NR^{a}R^{a}, —P(O)(OH)₂, —B(OH)₂, —CO₂R^{a}, —C(O)R^{a}, —CONR^{a}R^{a}, C₂₋₄-alkyl, —C₂₋₄-alkenyl, C₂₋₄-alkynyl and C₁₋₄-haloalkyl; wherein R^{a} is independently at each occurrence selected from H and C₁-C₄ alkyl, or a pharmaceutically acceptable salt thereof. It may be that the pendant group is, or comprises, a group selected from —COOH, —OH, —NH₂, —SH, —CH═CH₂, —C═CH, —S(O)₂F and —N₃.

Alternatively, or additionally, it may be that the polymer backbone comprises a heteroatom, an arylene, such as phenylene, a heteroarylene and/or an unsaturated hydrocarbon, e.g. an alkenylene or an alkynylene, within the main linear chain of the polymer backbone. Such polymers include, e.g. polyphenylene sulfide (PPS).

It may be that the polymer backbone comprises a biopolymer or a synthetic polymer.

It may be that the polymer backbone comprises a synthetic polymer.

It may be that the polymer backbone comprises a polycarbonate, a polyester, a polyphthalamide, a polyacrylate, a polyarylate, a polyamideimide, a polyimide, a polyamide, a polyolefin, a polyurethane, a chlorinated polyethylene, a linear hydrocarbon polymer (e.g. polyethylene), a poly(vinylnaphthalene), or a copolymer thereof. It may be that the polymer backbone comprises a polyurethane.

It may be that the polymer backbone comprises acrylonitrile butadienestyrene (ABS), polybutylene terephthalate (PBT), polyethylene terephthalate (PET), polyphenylene oxide, polyphenylene ether, polyphenylene sulfide, polystyrene, styrene-acrylonitrile resin (SAN), poly ether ketone (PEEK), poly(methyl methacrylate) (PMMA), polyvinyl acetate, polysulfone, polyether sulfone, polyether imide, polyvinyl chloride, polyethylene napthalate, polypropylene, polyvinyl toluene, polyvinylpyrrolidone (PVP), or a copolymer thereof.

It may be that the polymer backbone comprises a biopolymer. The biopolymer may be a polypeptide or a polysaccharide.

The polymer backbone may comprise a polypeptide. Typically, the polypeptide will be a protein, or a (partial) hydrolysis thereof. The protein may be keratin, elastin, or collagen. The (partial) hydrolysis may be gelatin, which may be referred to as hydrolysed collagen. The protein may be a glycoprotein, e.g. fibrinogen. The protein may be a proteoglycan, e.g. heparan sulfate proteoglycan, chondroitin sulfate proteoglycan or keratan sulfate proteoglycan.

The polymer backbone may comprise a polysaccharide. It may be that the compound of Formula (I) is covalently coupled to the polysaccharide via an ester bond. Alternatively, it may be that the compound of Formula (I) is covalently coupled to the polysaccharide via an amide bond.

It may be that the polysaccharide is a glucan, or a modified form thereof. Illustrative glucans include dextran, floridean starch, glycogen, pullulan, starch, cellulose, chrysolaminarin, curdlan, laminarin, lentinan, lichenin, oat beta-glucan, pleuran and zymosan. The glucan may be cellulose or a modified form thereof. The polysaccharide may be a modified polysaccharide, e.g. carboxymethylcellulose or carboxymethyl starch. It may be that the polysaccharide is a glycosaminoglycan.

It may be that the polysaccharide is selected from the group consisting of cellulose, carboxymethylcellulose, carboxymethyl starch, pectin, alginic acid, carrageenan, heparin, gellan gum, agarose, hyaluronic acid, chitosan or a pharmaceutically acceptable salt thereof.

The polysaccharide may be or may comprise an acidic polysaccharide. The acidic polysaccharide may comprise an acidic monosaccharide unit, such a uronic acid. Uronic acids include glucuronic acid, galacturonic acid, iduronic acid and mannuronic acid. It may be that the acidic polysaccharide comprises a glucuronic acid and/or mannuronic acid monosaccharide unit.

The acidic polysaccharide may be a glycosaminoglycan.

The acidic polysaccharide may be selected from the group consisting of heparin, chondroitin sulfate, dermatan sulfate, keratan sulfate, hyaluronic acid, gellan gum, pectin, heparan sulfate, alginic acid, carrageenan, carboxymethylcellulose and carboxymethyl starch, or a pharmaceutically acceptable salt thereof. It may be that the acidic polysaccharide is selected from the group consisting of carboxymethylcellulose, carboxymethyl starch, pectin, alginic acid, carrageenan, gellan gum and hyaluronic acid.

It may be that the acidic polysaccharide is alginic acid. It may be that the acidic polysaccharide is an alginate, e.g. calcium and/or sodium alginate.

The polymer backbone (and the modified polymer) will consist of individual structural units. It may be that greater than 1% of the structural units are covalently coupled to the compound of Formula (I).

### Modified Polymer

The modified polymer may comprise structural unit (A):

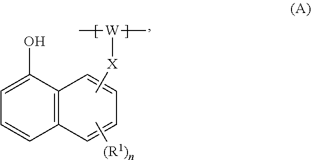

wherein W is a monosaccharide residue; and X, R¹ and n are as defined for Formula (I).

The modified polymer may comprises structural unit (Aa):

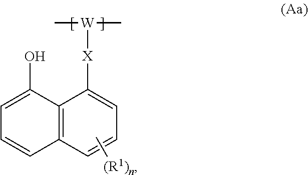

wherein W is a monosaccharide residue; and X, R¹ and n are as defined for Formula (I).

The modified polymer may comprise structural unit (Ai) and/or (Bi):

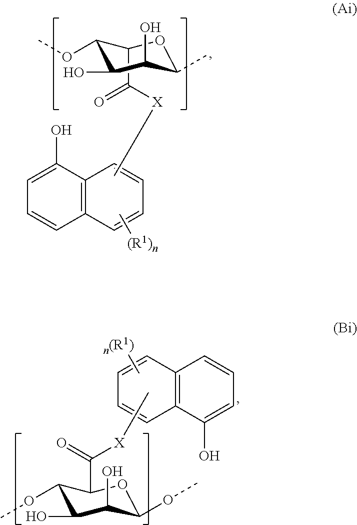

wherein X, R¹ and n are as defined above.

The modified polymer may comprise structural unit (Aii) and/or (Bii):

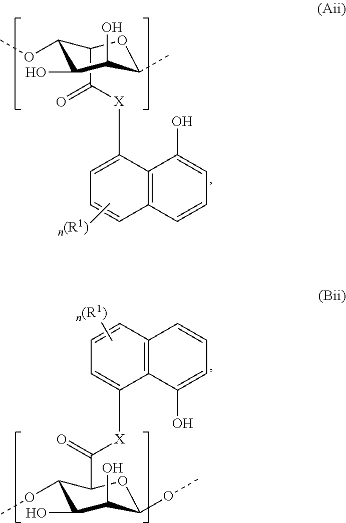

R¹ and n are as defined above.

The modified polymer may comprise structural unit (Aiii) and/or (Biii):

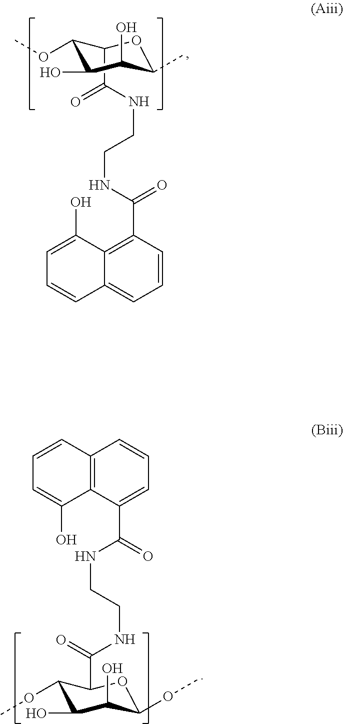

It may be that greater than 1% of the structural units of the modified polymer are structural unit (A), (Aa), (Ai), (Bi), (Aii), (Bii), (Aiii) or (Biii).

It may be that the modified polymer is a modified polymer matrix.

### Wound Dressing

The wound dressing will typically comprise an absorbent layer to be placed directly over the wound to be treated. The absorbent layer will allow wound exudate to be absorbed therein. It may be that the wound dressing comprises an absorbent layer, wherein the absorbent layer comprises (e.g. is formed from) the modified polymer. It may be that the enzymatic substrate is absorbed into the absorbent layer.

It may be that the wound dressing is semi-occlusive or occlusive. It may be that the wound dressing is semi-occlusive. For example, the wound dressing may comprise a (semi-)occlusive layer located proximate to said absorbent layer, such that the absorbent layer and wound to be treated may be protected from outside liquids.

It may be that the wound dressing comprises an antimicrobial agent, e.g. medical grade honey, iodine, silver, polyhexanide, dialkylcarbamoyl chloride or bismuth tribromophenate. It may be that the antimicrobial agent is silver, e.g. colloidal silver or silver nanoparticles.

It may be that the wound dressing is a hydrocolloid wound dressing. It may be that the wound dressing further comprises an adhesive layer for adhering the wound dressing to skin/tissue surrounding a wound. It may be that the wound dressing forms part of a pad of material (e.g. a gauze) for taping the wound dressing to a wound.

It may be that the wt % of the modified polymer is >50 wt % (of the total weight of the wound dressing). It may be that the wt % of the modified polymer is >75 wt %. It may be that the wt % of the modified polymer is >90 wt %, e.g. 95 or 97.5 wt %.

It may be that the wound dressing is for detecting the presence of bacteria, e.g. in a wound. The bacteria may be as defined below in connection with the third and fourth aspects of the invention.

### Enzymatic Substrate

In an embodiment, the wound dressing also comprises an enzymatic substrate of Formula (II),

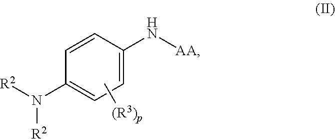

wherein AA is an amino acid bonded to the rest of the substrate via an amide bond;
R² is independently at each occurrence a C₁₋₅ alkyl;
p is 0, 1, 2 or 3; and
R³ is independently at each occurrence selected from halo, C₁₋₃ alkyl, C₂₋₃ alkenyl, C₁₋₃ haloalkyl, —O—C₁₋₃ alkyl, —O—C₁₋₃ haloalkyl and CN,
or a pharmaceutically acceptable salt thereof.

It may be that the enzymatic substrate is a substrate of Formula (IIa):

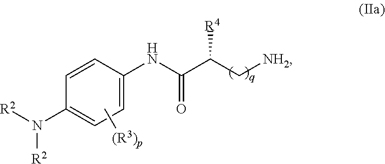

wherein R⁴ is selected from H and an amino acid side chain; q is 0 or 1; and R₂, R₃ and p are as defined above, or a pharmaceutically acceptable salt thereof.

It may be that R² is independently at each occurrence a C₁₋₃ alkyl. It may be that R² is independently at each occurrence a C₁₋₂ alkyl. It may be that R² is at each occurrence Me. It may be that R² is at each occurrence Et.

It may be that R³ is independently at each occurrence selected from halo and C₁₋₃ alkyl. p may be 0, 1 or 2. p may be 0 or 1. Preferably, p is 0.

It may be that R⁴ is selected from H, Me and i-Bu. It may be that R⁴ is i-Bu.

It may be that the enzymatic substrate is selected from:

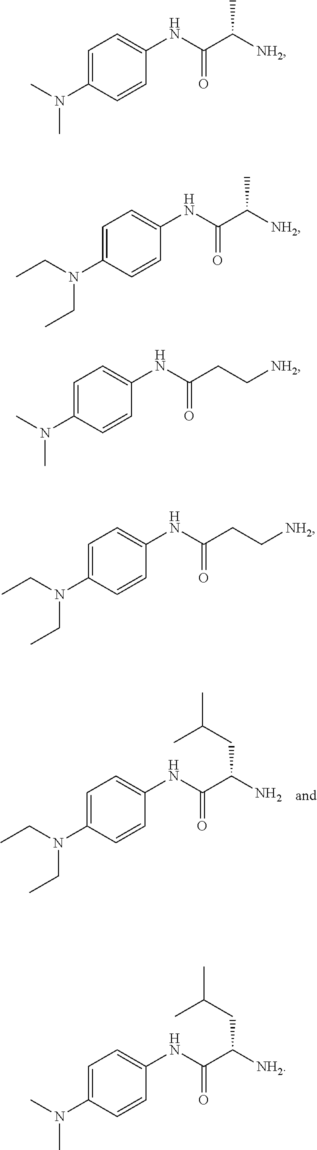 [SMILES: C[C@H](N)C(=O)Nc1ccc(N(C)C)cc1 ; CCN(CC)c1ccc(NC(=O)[C@H](C)N)cc1 ; CN(C)c1ccc(NC(=O)CCN)cc1 ; CCN(CC)c1ccc(NC(=O)CCN)cc1 ; CCN(CC)c1ccc(NC(=O)[C@@H](N)CC(C)C)cc1 ; CC(C)C[C@H](N)C(=O)Nc1ccc(N(C)C)cc1]

It may be that the enzymatic substrate is selected from:

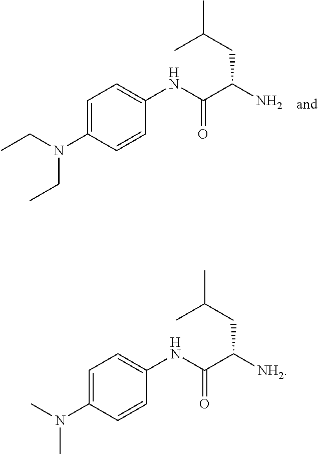 [SMILES: CCN(CC)c1ccc(NC(=O)[C@@H](N)CC(C)C)cc1 ; CC(C)C[C@H](N)C(=O)Nc1ccc(N(C)C)cc1]

It may be that the wound dressing comprises from 1 to 100 mg enzymatic substrate per g of modified polymer. It may be that the wound dressing comprises from 1 to 80 mg enzymatic substrate per g of modified polymer. It may be that the wound dressing comprises from 1 to 60 mg enzymatic substrate per g of modified polymer. It may be that the wound dressing comprises from 10 to 60 mg enzymatic substrate per g of modified polymer. It may be that the wound dressing comprises from 1 to 40 mg enzymatic substrate per g of modified polymer. It may be that the wound dressing comprises from 20 to 80 mg enzymatic substrate per g of modified polymer.

It may be that the wound dressing comprises from 1 to 100 mg enzymatic substrate per g of wound dressing. It may be that the wound dressing comprises from 1 to 80 mg enzymatic substrate per g of wound dressing. It may be that the wound dressing comprises from 1 to 60 mg enzymatic substrate per g of wound dressing. It may be that the wound dressing comprises from 10 to 60 mg enzymatic substrate per g of wound dressing. It may be that the wound dressing comprises from 1 to 40 mg enzymatic substrate per g of wound dressing. It may be that the wound dressing comprises from 20 to 80 mg enzymatic substrate per g of wound dressing.

The enzymatic substrate is not covalently coupled to the polymer backbone. Typically, the enzymatic substrate is absorbed into the wound dressing. It may be that the modified polymer is a modified polymer matrix and the enzymatic substrate is dispersed throughout the modified polymer matrix.

Once the wound dressing has been applied to a wound the enzymatic substrate is free to diffuse into the wound. Without wishing to be bound by theory, once the enzymatic substrate diffuses into a wound and encounters bacteria that produces an appropriate enzyme, a compound of Formula IIb;

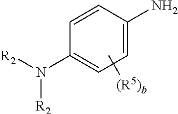

IIb, wherein R₂, R₃ and p are as defined above for Formula II), is released via enzymatic hydrolysis of the enzymatic substrate. The compound of Formula IIb is thought to diffuse back into the wound dressing where it reacts with the compound of Formula (I) to form a chromophore.

The presence of the chromophore causes a colour change. Where the chromophore absorbs wavelengths in the visible range, this may be observed by visual inspection of the dressing, e.g. when the wound dressing is changed. The wound dressing of the present invention therefore allows the presence of bacteria in a wound to be indicated by a colour change, e.g. a blue colour change. As the colour change is observed in situ, treatment may be initiated rapidly. In addition, the risk of outside contamination is reduced when compared to alternative bacterial detection systems that rely on swabbing and remote analysis.

Also disclosed herein is a reacted wound dressing comprising a modified polymer, wherein the modified polymer comprises a polymer backbone covalently coupled to a compound of Formula (V):

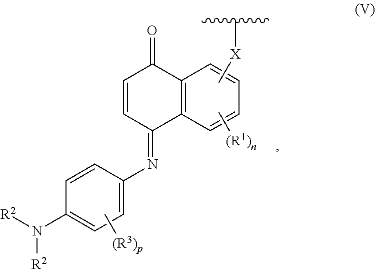

wherein the wound dressing, the polymer backbone, X, R¹, R², R³, n and p are as defined above. It may be that the compound of Formula (V) is the compound of Formula (Va):

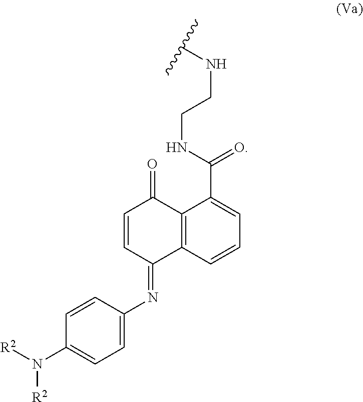

### Sterile Package

The second aspect of the invention provides a sterile package comprising a wound dressing according to the first aspect of the invention. It may be that the sterile package is a plastic or paper sterilization pouch (or peel pack) comprising said wound dressing. Packages may be sterilised by various methods, such as placing the package in a steam autoclave or via dry heat sterilization, unsaturated chemical-vapor sterilization, or gamma irradiation sterilization.

### Use/Method of Detection

In the uses and methods described herein, references to “a wound dressing according to the first aspect of the invention” (or to a “wound dressing according to an eighth aspect of the invention”) also includes such a wound dressing that has been removed from the sterile package of the second (or tenth) aspect of the invention. Accordingly, such uses and methods may also comprise a preliminary step of removing the wound dressing from the sterile package.

The third aspect of the invention provides a use of a wound dressing according to the first aspect (or eighth aspect) of the invention for detecting the presence of bacteria, e.g. in a wound.

The fourth aspect of the invention provides a method for detecting the presence of bacteria in a wound, the method comprising applying a wound dressing according to the first aspect (or eighth aspect) of the invention to a wound, removing the wound dressing from the wound and observing any colouration on the wound dressing. Colouration, e.g. blue coloration, on the wound dressing indicates the presence of bacteria.

The bacteria in the third or fourth aspect of the invention may be a Gram-negative strain of bacteria. The Gram-negative strain of bacteria may be a bacteria selected from Acinetobacter baumannii, Stenotrophomonas maltophilia, Escherichia coli and Pseudomonas aeruginosa. The Gram-negative strain of bacteria may be Escherichia coli or Pseudomonas aeruginosa.

The bacteria may be a Gram-positive strain of bacteria. The Gram-positive strain of bacteria may be Pseudomonas aeruginosa, Enterococcus faecalis, Streptococcus pyogenes, Staphylococcus aureus (e.g. Methicillin-resistant Staphylococcus aureus (MRSA)). The Gram-positive strain of bacteria may be Staphylococcus aureus.

It may be that the bacteria produces an aminopeptidase, such as L-alanyl aminopeptidase or L-Leucyl aminopeptidase.

The wound may be a chronic wound. Exemplary chronic wounds include a sloughy wound, a necrotic wound or a granulating wound.

The wound may comprise the bacteria at less than about 1×10¹⁰ CFU. It may be that the wound comprises the bacteria at less than about 1×10⁸ CFU. It may be that the wound comprises the bacteria at less than about 1×10⁶ CFU. It may be that the wound comprises the bacteria at less than about 1×10⁴ CFU. The wound may comprise the bacteria at greater than about 1×10³ CFU. It may be that the wound comprises the bacteria at greater than about 1×10⁴ CFU. The wound may comprise bacteria in a range from about 1×10³ CFU to about 1×10¹² CFU; e.g. the wound may comprise bacteria in a range from about 1×10⁴ CFU to about 1×10¹⁰ CFU.

A bacterial count of 1×10⁶ CFU is considered likely to go on to produce an infection in a wound. Exemplary wound dressings of the invention provide a bacteria detection threshold that is at or below this level, i.e. they provide a detectable colour change when contacted with a wound for a requisite period of time when the wound comprises at least 1×10⁶ CFU (or, e.g., comprises at least 1×10⁵ CFU for a detection threshold below said level).

The method of the fourth aspect of the invention may comprise removing the wound dressing from the wound at least about 12 h after applying the wound dressing. The method may comprise removing the wound dressing from the wound at least about 24 h after applying the wound dressing. The method may comprise removing the wound dressing from the wound at least about 48 h after applying the wound dressing. The method may comprise removing the wound dressing from the wound at least about 72 h after applying the wound dressing. The method may comprise observing any colouration on the surface of the wound dressing that was in contact with the wound. The coloration may be blue coloration.

A further aspect of the invention comprises use of a modified polymer as disclosed herein (for example a modified polymer of formula (I), or a modified polymer comprising any of structural unit (A), (Aa), (Ai) to (Aii), and (Bi) to (Biii)) in a wound dressing.

### Method of Forming Modified Polysaccharide

A fifth aspect of the invention provides a method of forming a modified polysaccharide comprising a modified monosaccharide residue of Formula (III):

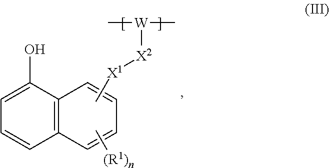

wherein X¹ is a covalent bond or a linker;
X² is selected from NH, O and S;
W is a monosaccharide residue;
R¹ is independently at each occurrence selected from halo, C₁₋₃ alkyl, C₂₋₃ alkenyl, C₁₋₃ haloalkyl, —O—C₁₋₃ alkyl, —O—C₁₋₃ haloalkyl and CN; and
n is 0, 1, 2 or 3,
the method comprising:
a) contacting an acidic polysaccharide with a compound of Formula (IV):

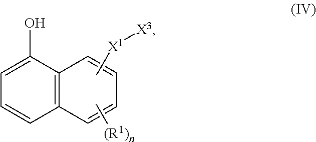

to form the modified polysaccharide,
wherein X³ is selected from —NH₂, —OH and —SH,
with the proviso that when X² is NH, X³ is —NH₂; when X² is O, X³ is —OH; and when X² is S, X³ is —SH.
   X¹, X² and X³

X¹ may be a covalent bond. Typically however, X¹ is a linker. The linker may be —Y—Z¹-L¹-, wherein Y is selected from —C(O)—, —S(O)₂— and —S(O)—; Z¹ is selected from O, S and NH; and L¹ is C₁₋₈ alkylene, C₂₋₈ alkenylene and C₂₋₈ alkynylene, wherein L¹ is optionally substituted where chemically possible with from 1 to 4 substituents selected from oxo, halo, —O—C₁₋₃ alkyl, —O—C₁₋₃ haloalkyl and CN. Y may be selected from —C(O)— and —S(O)₂—. Y may be —C(O)—. It may be that Z¹ is NH. It may be that L¹ is C₁₋₈ alkylene, e.g. C₁₋₄ alkylene. It may be that L, is ethylene. L, may be unsubstituted.

X² may be NH and X³ may be —NH₂.

### R¹ and n

It may be that R¹ is independently at each occurrence selected from halo and C₁₋₃ alkyl. It may be that n is 0, 1 or 2. It may be that n is 0 or 1. Preferably, n is 0.

### Step a)

It may be that step a) comprises suspending the acidic polysaccharide in a solvent to form a suspension. The solvent may be an aqueous solvent. For example, the solvent may comprise deionised water. The solvent may comprise an organic solvent. It may be that step a) further comprises adding a carboxyl activating agent to the suspension. Suitable carboxyl activating agents include carbodiimides and salts thereof. The carboxyl activating agent may be EDC, DCC, DIC or a hydrochloride salt thereof. The carboxyl activating agent may be EDC. The carboxyl activating agent may be EDC-HCl.

Step a) may comprise stirring the suspension at room temperature. It may be that the suspension is maintained at a pH of less than 7. It may be that the suspension is maintained at a pH of less than 5. It may be that the suspension is maintained at a pH of from 2 to 4. The pH of the suspension may be adjusted by the addition of a suitable acid, e.g. HCl.

Step a) may comprise adding the compound of Formula (IV) to the suspension to form a mixture. The compound of Formula (IV) may be first dissolved in a suitable organic solvent, such as DMSO or DMF, before being added to the suspension.

Step a) may comprise stirring the mixture at room temperature. The mixture may be stirred for 24 h or longer. The mixture may be stirred for 24-72 h. It may be that the mixture is maintained at a pH of less than 7. It may be that the mixture is maintained at a pH of less than 5. It may be that the mixture is maintained at a pH of from 2 to 4. The pH of the mixture may be adjusted by the addition of a suitable acid, e.g. HCl.

### Step aa)

The method may further comprise step aa); isolating the modified polysaccharide. Step aa) may comprise filtering the mixture to provide a filtrate comprising the modified polysaccharide. The filtrate may be washed with an organic solvent, e.g. ethanol. The filtrate may be resuspended in an organic solvent, e.g. ethanol, stirred, re-filtered and washed again with an organic solvent, e.g. ethanol. This process may be repeated until the compound of Formula (IV) is no longer detected within the filtrate (e.g. via TLC or HPLC evaluation). Step aa) may further comprise drying the modified polysaccharide, e.g. in a vacuum oven for 3-4 h.

### Modified Polysaccharides (C) and (E)

The method may be a method of forming modified polysaccharide (C):

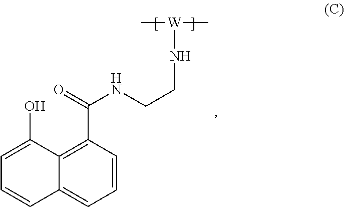

wherein the method comprises contacting the acidic polysaccharide with compound (D):

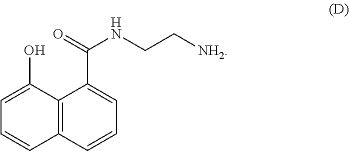 [SMILES: NCCNC(=O)c1cccc2cccc(O)c12]

Alternatively, the method may be a method of forming modified polysaccharide (E):

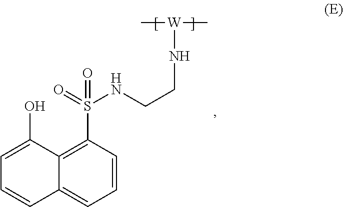

wherein the method comprises contacting the acidic polysaccharide with compound (F):

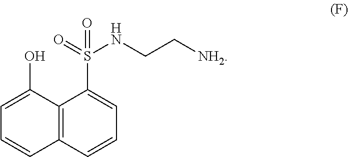 [SMILES: NCCNS(=O)(=O)c1cccc2cccc(O)c12]

### Acidic Polysaccharide

The acidic polysaccharide may comprise an acidic monosaccharide unit, such as a uronic acid. Uronic acids include glucuronic acid, galacturonic acid, iduronic acid and mannuronic acid. It may be that the acidic polysaccharide comprises a glucuronic acid and/or mannuronic acid monosaccharide unit.

The acidic polysaccharide may be a glycosaminoglycan.

The acidic polysaccharide may be selected from the group consisting of heparin, chondroitin sulfate, dermatan sulfate, keratan sulfate, hyaluronic acid, gellan gum, pectin, heparan sulfate, alginic acid, carrageenan, carboxymethylcellulose and carboxymethyl starch, or a pharmaceutically acceptable salts thereof.

It may be that the acidic polysaccharide is alginic acid. It may be that the acidic polysaccharide is an alginate, e.g. calcium and/or sodium alginate.

### W

W may be a uronic acid. For example, W may be glucuronic acid, galacturonic acid, iduronic acid or mannuronic acid. It may be that W is glucuronic acid. It may be that W is mannuronic acid.

### Enzymatic Substrate Deposition

The method may further comprise step b): depositing an enzymatic substrate of Formula (II) into the modified polysaccharide. The enzymatic substrate of Formula (II) may be as defined above in regard to the first aspect of the invention.

Step b) may comprise contacting the modified polysaccharide with a solution, e.g. an aqueous solution, comprising the enzymatic substrate to form a wet modified polysaccharide. Contacting the modified polysaccharide with the solution may comprise adding the solution dropwise to the modified polysaccharide to form the wet modified polysaccharide. Alternatively, contacting the modified polysaccharide with the solution may comprise adding the modified polysaccharide to a vessel containing the solution. The wet modified polysaccharide may then be dried.

The solution may comprise from 100 to 5000 mg/mL enzymatic substrate. The solution may comprise from 100 to 4000 mg/mL enzymatic substrate. The solution may comprise from 200 to 2000 mg/mL enzymatic substrate.

Step b) may comprise depositing from 1 to 100 mg enzymatic substrate of per g of modified polysaccharide. Step b) may comprise depositing from 1 to 75 mg enzymatic substrate per g of modified polysaccharide. Step b) may comprise depositing from 1 to 60 mg enzymatic substrate per g of modified polysaccharide. Step b) may comprise depositing from 10 to 60 mg enzymatic substrate per g of modified polysaccharide. Step b) may comprise depositing from 20 to 75 mg enzymatic substrate per g of modified polysaccharide.

### EXAMPLES

### General Synthesis

The skilled person will appreciate that adaptation of methods known in the art could be applied in the manufacture of the wound dressings, compounds, modified polymers, etc of the present invention.

For example, the skilled person will be immediately familiar with standard textbooks such as “Comprehensive Organic Transformations—A Guide to Functional Group Transformations”, RC Larock, Wiley-VCH (1999 or later editions); “March's Advanced Organic Chemistry—Reactions, Mechanisms and Structure”, MB Smith, J. March, Wiley, (5th edition or later); “Advanced Organic Chemistry, Part B, Reactions and Synthesis”, FA Carey, RJ Sundberg, Kluwer Academic/Plenum Publications, (2001 or later editions); “Organic Synthesis—The Disconnection Approach”, S Warren (Wiley), (1982 or later editions); “Designing Organic Syntheses” S Warren (Wiley) (1983 or later editions); “Heterocyclic Chemistry”, J. Joule (Wiley 2010 edition or later); (“Guidebook To Organic Synthesis” RK Mackie and DM Smith (Longman) (1982 or later editions), etc., and the references therein as a guide.

The skilled person is familiar with a range of strategies for synthesising organic and particularly heterocyclic molecules and these represent common general knowledge as set out in text books such as Warren “Organic Synthesis: The Disconnection Approach”; Mackie and Smith “Guidebook to Organic Chemistry”; and Clayden, Greeves, Warren and Wothers “Organic Chemistry”. Such strategies include “click chemistry”, which has been heavily covered in numerous books and journal articles, such as A. B. Lowe, Polym. Chem., 2020, 1,17-36; R. Hoogenboom, Angew. Chem. Int. Ed., 2010, 49, 3415-7; J. Dong, L. Krasnova, M. G. Finn, K. B. Sharpless,. Angew. Chem. Int. Ed., 2014, 53, 9430-9448; and F. Himo, T. Lovell, R. Hilgraf, V. V. Rostovtsev, L. Noodleman, K. B. Sharpless, V. V. Fokin, J. Am. Chem. Soc., 2005, 127, 210-216.

The skilled person will also be familiar with a range of strategies for synthesising modified derivatives of known polymers. Examples of such syntheses are described in F. Ilhan, et al., J. Am. Chem. Soc., 2000, 122, 5895-5896; F. Ilhan, et al., Macromolecules, 1999, 32, 6159-6162; F. Ilhan, et al., Macromolecules, 2001, 34, 2597-2601; A. K. Boal, et al., Tetrahedron, 2002, 58, 765-770; R. J. Thibault, et al., J. Am. Chem. Soc., 2002, 124, 15249-15254; R. J. Thibault, et al., J. Am. Chem. Soc., 2003, 125, 11249-11252; and J. B. Carroll, et al., Lett. Org. Chem., 2004, 1, 227-230.

Polysaccharides, e.g. cellulose, may be modified as described in Ma, H., Hsiao, B.S., Chu, B. (2016). Modified Cellulose. In: Drioli, E., Giorno, L. (eds) Encyclopedia of Membranes. Springer, Berlin, Heidelberg:

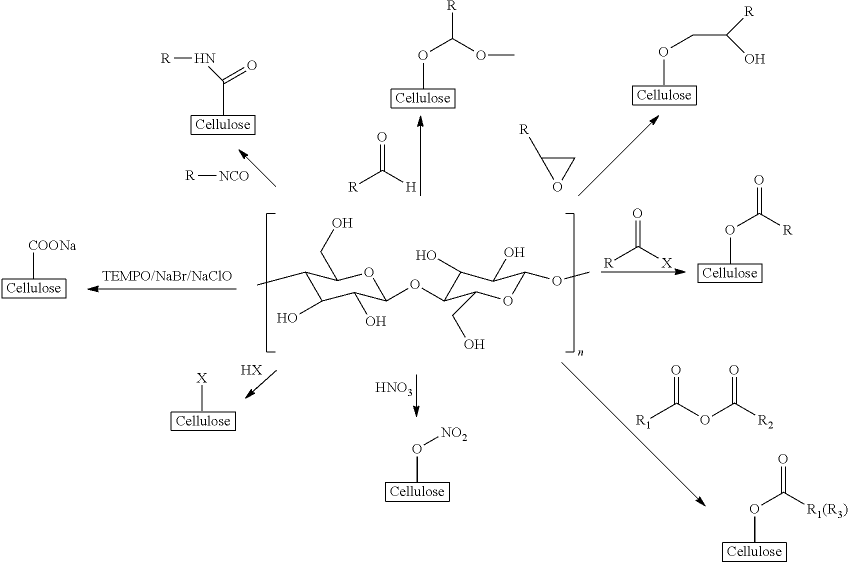

The skilled chemist will exercise his/her judgement and skill as to the most efficient sequence of reactions for synthesis of a given target compound and will employ protecting groups as necessary. This will depend inter alia on factors such as the nature of other functional groups present in a particular substrate. Clearly, the type of chemistry involved will influence the choice of reagent that is used in the said synthetic steps, the need, and type, of protecting groups that are employed, and the sequence for accomplishing the protection/deprotection steps. These and other reaction parameters will be evident to the skilled person by reference to standard textbooks and to the examples provided herein.

Sensitive functional groups may need to be protected and deprotected during synthesis of a compound of the invention. This may be achieved by conventional methods, for example as described in P. G. M. Wuts, Greene's Protective Groups in Organic Synthesis, John Wiley & Sons, Inc., Michigan, 2014, and references therein.

All commercially available chemical reagents and solvents were obtained from Sigma-Aldrich, Apollo Scientific, Alfa Aesar, Fluorochem, or Fischer Scientific and were used without further purification. Melting points were recorded on a Reichart-Kofler hot-stage microscope apparatus and are uncorrected. Infrared spectra were recorded in the range 4000-600 cm⁻¹ using a Perkin Elmer Spectrum BX FT-IR spectrophotometer. NMR spectra were obtained using a Bruker Ultrashield 300 spectrometer at 300 MHz for ¹H spectra or at 75 MHz for ¹³C spectra. High resolution accurate mass measurements were collected by the EPSRC National Mass Spectrometry Facility at Swansea University. Low-resolution mass spectra were recorded on a Bruker Esquire 3000 plus analyser using an electrospray source in either positive or negative ion mode. Thin layer chromatography was performed on Merck silica gel 60F₂₅₄ and the results were analysed by UV lamp (254 nm and 265 nm), and/or by the addition of ninhydrin stain (amine, amide content). Fischer silica gel 60 (35-70 micron) was used for column chromatography; the samples were pre-absorbed onto silica 60 (35-70 micron). Flash column chromatography was performed using a CombiFlash® NextGen 100 system using RediSep Gold® normal-phase silica for normal phase chromatography and RediSep Gold® C18 for reverse phase chromatography.

### Synthesis of Enzymatic Substrates

Enzymatic substrates of Formula (IIa) can be made according to the syntheses disclosed in M. Cellier, et al., Bioorg. Med. Chem., 22, 2014, 5249-5269; DE2646033A1; EP2292613A1; and U.S. Pat. No. 6,653,312B1 A. Enzymatic substrates of Formula (IIa) can also be made by methods according to, or analogous to, the methods described in Scheme 1 or the specific syntheses of enzymatic substrates 3a or 3b.

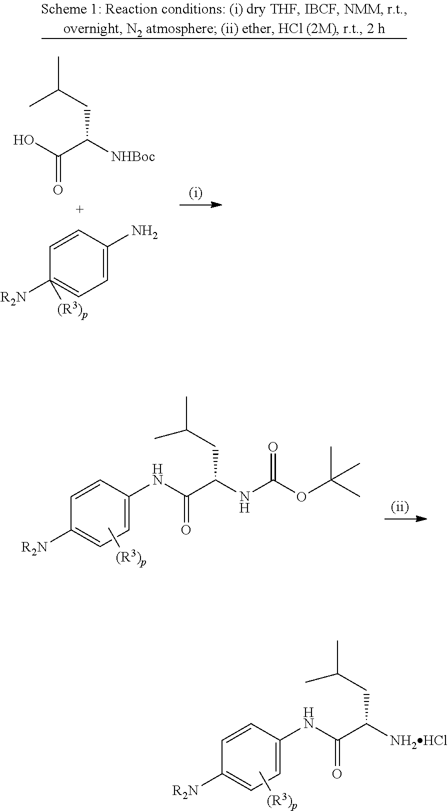

### Tert-butyl (1-{[4-(dimethylamino)phenyl]amino}-4-methyl-1-oxopentan-2-yl)carbamate (2a)

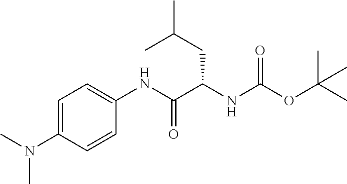 [SMILES: CC(C)C[C@H](NC(=O)OC(C)(C)C)C(=O)Nc1ccc(N(C)C)cc1]

Boc-L-leucine (1.02 g, 4.41 mmol, 1.2 eq.) was dissolved in 20 mL anhydrous THF under inert atmosphere in an ice bath. NMM (0.49 mL, 4.41 mmol, 1.2 eq.) and IBCF (0.58 mL, 4.41 mmol, 1.2 eq.) were added dropwise to the solution and the mixture was stirred at 0° C. for 30 minutes. Dimethyl-4-phenylenediamine (1a) (0.50 g, 3.67 mmol, 1.0 eq.) was added to the reaction mixture, and the ice bath removed after 30 mins. The reaction was stirred at room temperature overnight. The solvent was evaporated under reduced pressure and the crude product was resuspended in DCM, followed by extraction with 0.1 M aq. citric acid solution (2×30 mL), then 10% aq. NaHCO₃ solution (3×30 mL). The organic layer was washed with water (30 mL), and brine (30 mL), and dried over MgSO₄. The solvent was evaporated under vacuum and the crude product was purified by flash column chromatography (silica gel solid phase, gradient solvent method with petroleum ether and EtOAc mobile phase) to obtain 2a as a white powder (0.62 g, 1.8 mmol, 59%).

m.p.: 144-145° C.; ¹H NMR (300 MHz, CDCl₃): δ 8.12 (s, 1H, 6-H), 7.34 (d, Jₒ=9.1 Hz, 2H, 3-H), 6.66 (d, Jₒ=9.0 Hz, 2H, 2-H), 5.11 (d, J_{HH}=8.3 Hz, 1H, 12-H), 4.31-4.18 (m, 1H, 8-H), 2.89 (s, 6H, 5-H), 1.88-1.54 (m, 3H, 9-H, 10-H), 1.44 (s, 9H, 15-H), 0.96 (d, J_{HH}=6.4 Hz, 11-H), 0.95 (d, J_{HH}=6.2 Hz, 11′-H) ppm; ¹³C NMR (75 MHz, CDCl₃): δ 170.4 (7-C), 156.1 (13-C), 148.0 (4-C), 127.8 (1-C), 121.6 (3-C), 113.0 (2-C), 80.3 (14-C), 77.43, 77.20, 77.00, 76.58, 53.7 (8-C), 41.1 (9-C), 40.9 (5-C), 28.3 (15-C), 24.8 (10-C), 22.0 (11-C, 11′-C) ppm; IR (uₘₐₓ/cm⁻¹): 3327 (N—H), 2956 (C—H), 1685 (C═O), 1653 (C═O), 1519 (N—H), 1249 (C—N), 1165 (C—O); LRMS (ESI) m/z: 350.2 (MH+); HRMS (ESI) m/z found: 350.2446 (MH+); Calcd. for C₁₉H₃₁N₃O₃, 349.2365 and C₁₉H₃₂N₃O₃⁺, 350.2438.

### Tert-butyl (1-{[4-(diethylamino)phenyl]amino}-4-methyl-1-oxopentan-2-yl)carbamate (2b)

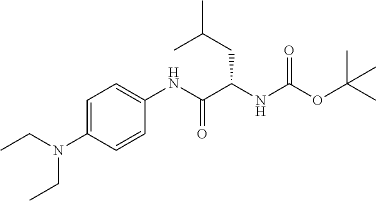 [SMILES: CCN(CC)c1ccc(NC(=O)[C@H](CC(C)C)NC(=O)OC(C)(C)C)cc1]

Boc-L-leucine (1.02 g, 4.41 mmol, 1.2 eq.) was dissolved in 20 mL anhydrous THF under inert atmosphere in an ice bath. NMM (0.49 mL, 4.41 mmol, 1.2 eq.) and IBCF (0.58 mL, 4.41 mmol, 1.2 eq.) were added dropwise to the solution and the mixture was stirred at 0° C. for 30 minutes. Diethyl-4-phenylenediamine (1b) (0.50 g, 3.67 mmol, 1.0 eq.) was added to the reaction mixture, and the ice bath removed after 30 mins. The reaction was stirred at room temperature overnight. The solvent was evaporated under reduced pressure and the crude product was resuspended in DCM, followed by extraction with 0.1 M aq. citric acid solution (2×30 mL), then 10% aq. NaHCO₃ solution (3×30 mL). The organic layer was washed with water (30 mL), and brine (30 mL), and dried over MgSO₄. The solvent was evaporated under vacuum and the crude product was purified by flash column chromatography (silica gel solid phase, gradient solvent method with petroleum ether and EtOAc mobile phase) to obtain 2b as a white powder (0.91 g, 2.7 mmol, 75%).

m.p.: 130-132° C.; ¹H NMR (300 MHz, CDCl₃) δ 7.93 (s, 1H, 7-H), 7.31 (d, Jₒ=9.1 Hz, 2H, 3-H), 6.62 (d, Jₒ=9.1 Hz, 2H, 2-H), 5.02 (d, J_{HH}=8.3 Hz, 1H, 13-H), 4.24-4.18 (m, 1H, 9-H), 3.31 (q, J_{HH}=7.1 Hz, 4H, 5-H), 1.82-1.51 (m, 3H, 10-H, 11-H), 1.45 (s, 9H, 16-H), 1.12 (t, J_{HH}=7.0 Hz, 4H), 0.96 (d, J_{HH}=6.4 Hz, 12-H), 0.95 (d, J_{HH}=6.2 Hz, 12′-H) ppm; ¹³C NMR (75 MHz, CDCl₃) δ 170.2 (8-C), 156.0 (14-C), 145.2 (4-C), 126.6 (1-C), 122.0 (3-C), 112.5 (2-C), 80.3 (15-C), 53.7 (9-C), 44.6 (5-C), 41.1 (11-C), 28.3 (16-C), 24.8 (10-C), 22.9 (12-C, 12′-C), 22.05, 12.5 (6-C) ppm; IR (umax/cm-1): 3290 (N—H), 2966 (C—H), 1653 (C═O), 1514 (N—H), 1249 (C—N), 1165 (C—O); LRMS (ESI) m/z: 378.3 (MH+); HRMS (ESI) m/z found: 378.2757 (MH+); Calcd. for C₂₁H₃₅N₃O₃, 377.2678 and C₂₁H₃₆N₃O₃⁺, 378.2751.

### (2S)-2-amino-N-[4-(dimethylamino)phenyl]-4-methyl-pentanamide hydrochloride (3a)

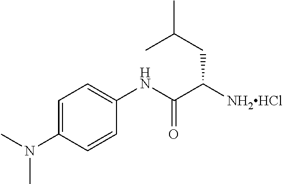

The N-Boc protected amide 2a (1.0 eq.) was stirred at room temperature in a 2M HCl solution in diethyl ether (10.0 eq.) for 2 h. After the reaction was complete the mixture was filtered and washed with diethyl ether.

m.p.: 248° C. (literature: 193-197° C.¹¹); ¹H NMR (300 MHz, MeOD): δ 7.89 (d, Jₒ=9.2 Hz, 2H, 3-H), 7.69 (d, Jₒ=9.2 Hz, 2H, 2-H), 4.16-4.11 (m, 1H, 8-H), 3.29 (s, 6H, 5-H), 1.88-1.72 (m, 3H, 10-H, 9-H), 1.042 (d, J_{HH}=6.3 Hz, 3H, 11-H), 1.039 (d, J_{HH}=6.3 Hz, 3H, 11′-H) ppm; ¹³C NMR (75 MHz, MeOD): δ 169.6 (7-C), 140.9 (1-C), 139.9 (4-C), 122.6 (2-C), 122.5 (3-C), 53.9 (8-C), 47.3 (5-C), 41.7 (9-C), 25.6 (10-C), 23.3 (11′-C), 22.2 (11-C) ppm; IR (uₘₐₓ/cm⁻¹): 2956 (C—H), 2870 (C—H), 1693 (C═O), 1514 (N—H), 1261 (C—N), 1116 (C—N); LRMS (ESI) m/z: 250.2 (MH+); HRMS (ESI) m/z found: 250.1917 (MH+); Calcd. for C₁₄H₂₃N₃O, 249.1841 and C₁₄H₂₄N₃O⁺, 250.1914.

### (2S)-2-amino-N-[4-(diethylamino)phenyl]-4-methyl-pentanamide hydrochloride (3b)

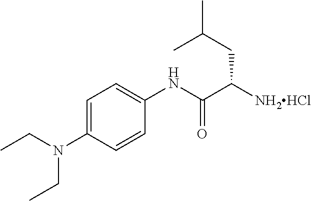

The N-Boc protected amide 2b (1.0 eq.) was stirred at room temperature in a 2M HCl solution in diethyl ether (10.0 eq.) for 2 h. After the reaction was complete the mixture was filtered and washed with diethyl ether.

m.p.: 245-248° C.; ¹H NMR (300 MHz, MeOD): δ 7.91 (d, Jₒ=9.1 Hz, 2H, 3-H), 7.62 (d, Jₒ=9.1 Hz, 2H, 2-H), 4.17 (t, J_{HH}=7.1 Hz, 1H, 9-H), 3.67 (q, J_{HH}=7.2 Hz, 4H, 5-H), 3.52 (q, J=7.0 Hz, 4H), 1.16 (t, J_{HH}=7.2 Hz, 6H, 6-H) ppm; ¹³C NMR (75 MHz, MeOD): δ 169.9 (8-C), 140.8 (1-C), 134.3 (4-C), 124.5 (2-C), 123.1 (3-C), 55.1 (5-C), 53.9 (9-C), 41.6 (10-C), 27.3, 25.5 (11-C), 23.3 (12′-C), 22.3 (12-C), 10.8 (6-C) ppm; IR (uₘₐₓ/cm⁻¹): 2956 (C—H), 2877 (C—H), 1693 (C═O), 1508 (N—H), 1257 (C—N); LRMS (ESI) m/z: 278.2 (MH+); HRMS (ESI) m/z found: 278.2231 (MH+); Calcd. for C₁₆H₂₇N₃O, 277.2154 and C₁₆H₂₈N₃O⁺, 278.2227.

### Synthesis of α-Naphthol Derivatives

α-naphthol derivatives (e.g. compounds of Formula (IV)) can be made by methods according to, or analogous to, the synthesis of α-naphthol derivative 8 or 10.

4-amino-1-(8-hydroxynaphthalen-1-yl)butan-1-one (8)

The amide type 1-naphthol derivative (8) was synthesised on a gram scale from 1,8-naphthalic anhydride (4) with an overall of 38% yield, using low-cost, commercially available materials and simple reaction conditions according to Scheme 2.

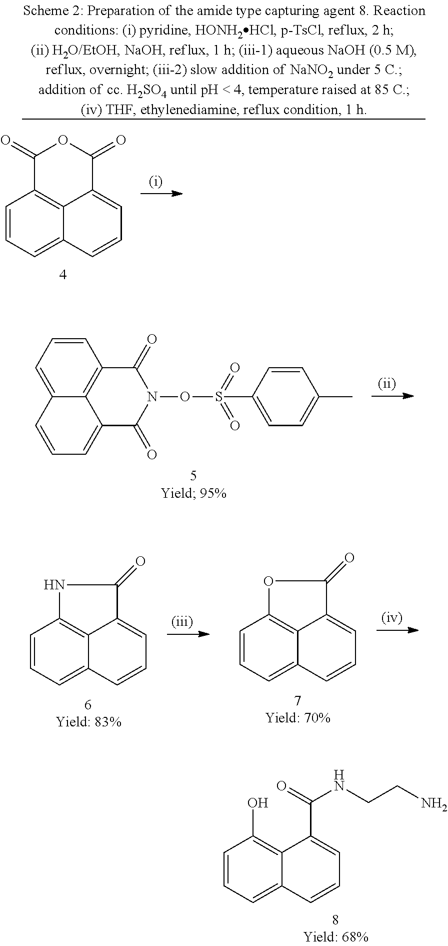 [SMILES: O=C1OC(=O)c2cccc3cccc1c23 ; Cc1ccc(S(=O)(=O)ON2C(=O)c3cccc4cccc(c34)C2=O)cc1 ; O=C1Nc2cccc3cccc1c23 ; O=C1Oc2cccc3cccc1c23 ; NCCNC(=O)c1cccc2cccc(O)c12]

### (1,3-dioxobenzo[de]isoquinolin-2-yl) 4-methylbenzenesulfonate (5)

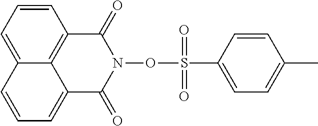 [SMILES: Cc1ccc(S(=O)(=O)ON2C(=O)c3cccc4cccc(c34)C2=O)cc1]

10.00 g Naphthalic anhydride (4) (50.0 mmol, 1 eq.) and 3.54 g hydroxylamine hydrochloride (50.0 mmol, 1 eq.) were added to 60 mL pyridine and the mixture was refluxed for one hour. The heating was then stopped and 21.00 g p-toluenesulfonyl chloride (100.0 mmol, 2 eq.) was added in four portions over 15 minutes. The heating was resumed for one more hour after which time the mixture was added to distilled water (250 mL), the precipitate was filtered, washed with 0.1 M NaOH solution (3×100 mL) and with water (100 mL) to obtain 5 (18.00 g, 48.5 mmol, 97%) as a yellow powder.

m.p.: 228-229° C. (literature: 228-229° C.¹²); ¹H NMR (400 MHz; DMSO-d₆): δ 8.55 (dd, 2H, Jₒ=8.24 Hz, Jₘ=0.92 Hz, Ar—H), 8.50 (dd, 2H, Jₒ=7.33 Hz, Jₘ=0.92 Hz, Ar—H), 7.97-7.89 (m, 4H, Ar—H), 7.53 (d, 2H, Jₒ=8.24 Hz, Ar—H), 2.48 (s, 3H, 12-H) ppm; ¹³C NMR (75 MHz, DMSO-d₆): δ 160.1 (C-7), 147.1 (Ar—C), 136.0 (Ar—C), 132.1 (Ar—C), 132.1 (Ar—C), 132.0 (Ar—C), 130.6 (Ar—C), 129.5 (Ar—C), 128.0 (Ar—C), 127.2 (Ar—C), 122.4 (Ar—C), 21.8 (12-C) ppm; IR (uₘₐₓ/cm⁻¹): 3098 (C—H), 3061 (C—H), 1699 (C═O), 1176 (S=O); LRMS (ESI) m/z: 368.1 (MH+); HRMS (ESI) m/z found: 368.0588 (MH+); Calcd. for C₁₉H₁₃NO₅S, 367.0514 and C₁₉H₁₄NO₅S⁺, 368.0587.

### (1H-benzo[cd]indol-2-one) (6)

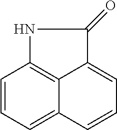 [SMILES: O=C1Nc2cccc3cccc1c23]

A mixture of 17.00 g compound 5 (46.0 mmol, 1 eq.) and 10.00 g NaOH (250.0 mmol, 5.5 eq.) was stirred in a mixture of distilled water and ethanol (200 mL, 3:1) for one hour under reflux. The heating was continued for one more hour during which time ethanol was allowed to evaporate. The mixture was cooled and acidified with concentrated HCl (30 mL) which allowed for CO₂ to evolve and the product to precipitate. The crude product was filtered obtaining 6 (6.5 g, 38.2 mmol, 83%) as a yellow powder (6.5 g, 38.2 mmol, 83%).

m.p.: 180° C. (literature: 180-182° C.¹³); ¹H NMR (400 MHz; DMSO-d6): δ 10.80 (s, 1H, 9-H), 8.18 (d, 1H, Jₒ=7.79 Hz, Ar—H), 8.03 (d, 1H, Jₒ=6.87 Hz, Ar—H), 7.78 (dd, Jₒ=8.0 Hz, Jₒ=7.0 Hz, 1H, Ar—H), 7.61 (d, 1H, Jₒ=8.24 Hz, Ar—H), 7.48 (dd, Jₒ=8.4 Hz, Jₒ=7.0 Hz, 1H, Ar—H), 7.00 (d, 1H, Jₒ=6.87 Hz, Ar—H) ppm; ¹³C NMR (75 MHz; DMSO-d₆): δ 169.4 (10-C), 138.6 (Ar—C), 131.4 (Ar—C), 129.6 (Ar—C), 129.5 (Ar—C), 129.4 (Ar—C), 127.4 (Ar—C), 126.2 (Ar—C), 124.2 (Ar—C), 119.9 (Ar—C), 106.6 (Ar—C) ppm; IR (uₘₐₓ/cm⁻¹): 3067 (N—H), 3089 (C—H), 1716 (C═O), 1494 (N—H); HRMS (ESI) m/z found: 170.0606 (MH+); LRMS (ESI) m/z: 170.1 (MH+); Calcd. for C₁₁ H₇NO, 169.0528 and C₁₁ H₈NO⁺, 170.0600.

### 2-oxatricyclo[6.3.1.0\hat{ }{4,12}]dodeca-1(11),4,6,8(12),9-pentaen-3-one (7)

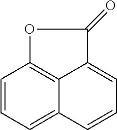 [SMILES: O=C1Oc2cccc3cccc1c23]

5.00 g 6 (29.6 mmol, 1.2 eq.) was dissolved in 0.5 M NaOH (aq.) solution (200 mL) and the mixture was stirred under reflux conditions overnight. The mixture was cooled using an ice bath and was kept under 5° C. 1.74 g NaNO₂ (25.00 mmol, 1 eq.) was added to the solution and was stirred for 10 minutes. In a conical flask ~100 mL water was set under pH 2 with cc. H₂SO₄ and was chilled on an ice bath until the solution was under 5° C. The reaction mixture was slowly added to the acidic solution under controlled pH and temperature until the addition was complete and the crude product crashed out from the solution. The resulting mixture was then heated slowly to reach 85° C. which allowed for N₂ to evolve. The mixture was stirred at room temperature for two hours after which time the precipitate was filtered off and washed with water. The product was dried giving 7 as a brown solid (3.80 g, 22.5 mmol, 76%).

m.p.: 105-108° C. (literature: 105-107.5° C.¹⁴); ¹H NMR (300 MHz, DMSO-d₆): δ 8.40 (d, Jₒ=8.2 Hz, 1H, Ar—H), 8.29 (d, Jₒ=7.1 Hz, 1H, Ar—H), 7.96 (dd, Jₒ=8.0 Hz, Jₒ=7.2 Hz, 1H, Ar—H), 7.85 (d, Jₒ=8.5 Hz, 1H, Ar—H), 7.69 (dd, Jₒ=8.4 Hz, Jₒ=7.4 Hz, 1H, Ar—H), 7.37 (d, Jₒ=7.3 Hz, 1H, Ar—H) ppm; ¹³C NMR (75 MHz, DMSO-d₆): δ 167.0 (9-C), 149.8 (Ar—C), 133.1 (Ar—C), 130.6 (Ar—C), 130.0 (Ar—C), 129.9 (Ar—C), 128.8 (Ar—C), 127.2 (Ar—C), 121.6 (Ar—C), 120.7 (Ar—C), 106.6 (Ar—C) ppm; IR (uₘₐₓ/cm⁻¹): 3072 (C—H), 1774 (C═O), 1226 (C—O); LRMS (ESI) m/z: 171.0 (MH+); HRMS (ESI) m/z found: 171.0445 (MH+); Calcd. for C₁₁ H₆O₂, 170.0368 and C₁₁ H₇O₂⁺, 171.0441.

### 4-amino-1-(8-hydroxynaphthalen-1-yl)butan-1-one (8)

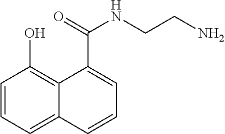 [SMILES: NCCNC(=O)c1cccc2cccc(O)c12]

Ethylenediamine (1.60 mL, 23.91 mmol, 4.0 eq.) and compound 7 (1.02 g, 5.98 mmol, 1.0 eq.) were dissolved in 30 mL anhydrous THF. The mixture was refluxed for 1 h, filtered and washed with THF and the resulting product was crystallised from methanol to obtain 8 as a pink powder (0.92 g, 4.00 mmol, 68%).

m.p.: 216-217° C. (literature: 200-201° C.¹⁵); ¹H NMR (300 MHz, DMSO-d₆): δ 8.15 (brs, 1H, 9-H), 7.83 (dd, Jₒ=8.2 Hz, Jₘ=1.3 Hz, 1H, Ar—H), 7.40 (dd, Jₒ=8.2 Hz, Jₒ=7.0 Hz, 1H, Ar—H), 7.36-7.20 (m, 3H, Ar—H), 6.82 (dd, Jₒ=8.8 Hz, Jₘ=1.4 Hz, 1H, Ar—H), 3.31 (t, 2H, J_{HH}=6.03 Hz, 12-H), 2.75 (t, 2H, J_{HH}=6.03 Hz, 13-H) ppm; ¹³C NMR (75 MHz, DMSO-d₆): δ 172.4 (C-10), 155.1 (Ar—C), 135.7 (Ar—C), 134.9 (Ar—C), 129.2 (Ar—C), 127.4 (Ar—C), 125.8 (Ar—C), 124.8 (Ar—C), 121.8 (Ar—C), 118.3 (Ar—C), 110.9 (Ar—C), 42.9 (12-C), 41.5 (13-C) ppm; IR (uₘₐₓ/cm⁻¹): 3263 (N—H), 2970 (C—H), 1639 (C═O), 1552 (N—H), 1271 (C—N), 1012 (C—O); LRMS (ESI) m/z: 230.3 (MH+); HRMS (ESI) m/z found: 231.1131 (MH+); Calcd. for C₁₃H₁₄N₂O₂, 230.1055 and C₁₃H₁₅N₂O₂*, 231.1128.

### N-(2-aminoethyl)-8-hydroxynaphthalene-1-sulfonamide (10)

The sulphonamide type capturing agent (10) was synthesised in a one-step reaction, using naphthosultone (9) as a starting material on a gram scale with 80% yield according to Scheme 3.

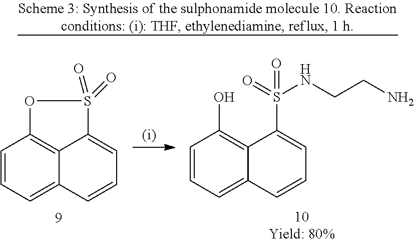 [SMILES: O=S1(=O)Oc2cccc3cccc1c23 ; NCCNS(=O)(=O)c1cccc2cccc(O)c12]

### N-(2-aminoethyl)-8-hydroxynaphthalene-1-sulfonamide (10)

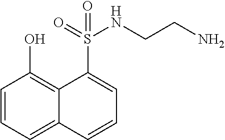 [SMILES: NCCNS(=O)(=O)c1cccc2cccc(O)c12]

To a solution of naphthosultone (9) (0.97 mmol) in THF (4.95 mL) was added ethylenediamine (3.88 mmol) and the solution was refluxed for one hour. After removing THF under reduced pressure, the product was precipitate by adding ethanol and filtered. Yellow solid (207 mg, 80%).

m.p.: 120° C.; ¹H-NMR (300 MHz; d₆-DMSO) δ: 8.06 (1 H, d, J=7.35 Hz, CH—Ar), 7.98 (1H, d, J=7.72 Hz, CH—Ar), 7.48-7.30 (3H, m, CH—Ar), 6.91 (1H, d, J=6.78 Hz, CH—Ar), 2.95-2.87 (2H, m, CH₂), 2.78-2.65 (2H, m, CH₂); IR (uₘₐₓ/cm⁻¹): 3510.45w (NH), 3300 broad (OH), 2970s (CH₂), 1519.91s (C=C), 1276.88s (SO₂).

### Modified Polysaccharide

Modified polysaccharides of the present invention may be prepared via methods according to, or analogous to, the method described in Scheme 4.

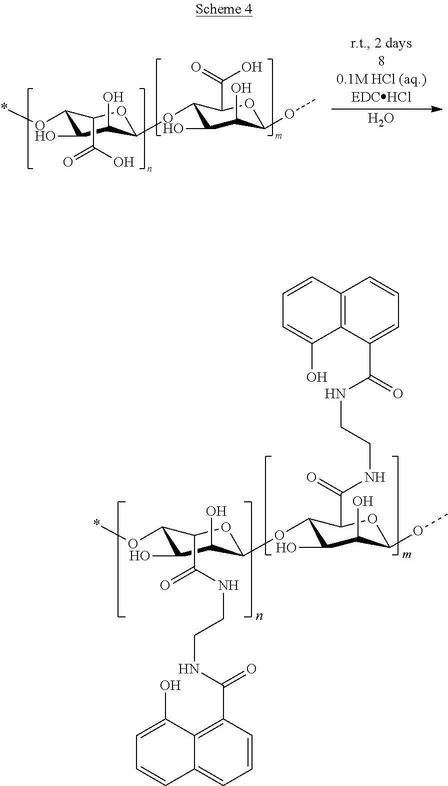

### Wound Dressings

Exemplary wound dressings of the present invention may be prepared via methods according to, or analogous to, the method described in Scheme 4, wherein

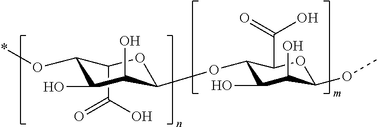

forms part of a wound dressing. The wound dressings of the present invention may be prepared via methods according to, or analogous to, the method described in ‘Modification of ActivHeal® Alginate’, below.

### Modification of ActivHeal® Alginate

ACTIVHEAL® simply A (10×20 cm) non-adhesive foam samples were provided by Advanced Medical Solutions Ltd. The wound dressings were cut into ~5-10×5-10 mm squares, using an ethanol washed pair of scissors. A maximum of 0.20 g of the material was added into 10 mL DI water, and the pH was adjusted to ~3 by the addition of 0.1 M aq. HCl solution. The solution was stirred for 10 mins at room temperature, followed by the addition of 0.33 g EDC •HCl (1.7 mmol) and the pH was maintained at ~3 by the addition of 0.1 M aq. HCl solution. The solution was stirred at room temperature for 10 mins followed by the addition of 0.88 g compound 8 which was dissolved in DMSO or DMF. The mixture was stirred at room temperature for 48 h. The wound dressings were filtered and were washed with ethanol. To ensure the full removal of any non-coupled amide (8), the foam samples were resuspended in ethanol, stirred for 10 mins and filtered, and this process was repeated until the amide (8) was not detectable from the filtrate (this could be maintained by TLC or HPLC evaluation). The samples were dried in a vacuum oven for 3-4 h.

The aqueous solution of 3a or 3b was prepared by the following: the desired amount of the enzymatic substrate (3.0 mg for 300 mg/L, 6.0 mg for 600 mg/L, 9.0 mg for 900 mg/L, 15.0 mg for 1500 mg/L, and 30.0 mg for 3000 mg/L) was weighed out on a 4 decimal place balance within ±0.3 mg error limit into a 10 mL volumetric flask and was adjusted to 10.0 mL with DI water. The solutions were absorbed into the foam samples and dried in a vacuum oven at 40° C. overnight.

### Microbiology

All media and reagents applied for the microbiology evaluations were obtained from either Fisher scientific (Loughborough, UK) or Sigma Aldrich (Dorset, UK) and of general laboratory grade unless otherwise stated in the text.

All bacterial strains used are produced by the National Collection of Type cultures and are publicly available. They are summarised in

### Table.

TABLE 1
Microbial strains used for the evaluation of the modified wound dressings.

| | Culture collection | |
|---|---|---|
| Bacterial strain | identifier | Reference |
| Escherichia coli - EC | NCTC 12241 | V. J. Boyle, et al., Antimicrob. |
| | | Agents Chemother., 1973, 3(3), |
| | | 418-424 |
| Pseudomonas aeruginosa - | NCTC 10662 | P. M. Waterworth, J. Med. Lab. |
| PSA | | Technol., 1969, 26(2), 106-110 |
| Staphylococcus aureus - SA | NCTC 6571 | N. G. Heatley, Biochem. J., 1944; |
| | | 38(1), 61-65 |

All cultures were maintained at −80° C. on Microbank Cryobeads (Pro-Lab Diagnostics, CA). Cultures were revived from freezer stocks to Tryptic Soy Agar (TSA) working stock plates.

Bacterial strains were routinely cultured from working stocks in Tryptic Soy Broth (TSB) and incubated at 37° C. overnight with shaking at 150 RPM. Overnight cultures were centrifuged at 3000×g for 5 minutes, the supernatant discarded, and the pellet resuspended in 10 mL phosphate buffered saline (PBS). Bacterial inoculums were standardised by optical density at 600 nm on an Amersham Biosciences Ultrospec 10 spectrophotometer (Amersham Biosciences, Amersham, UK) to contain between 1 and 3×10⁸ CFU/mL.

Lower concentration bacterial solutions were prepared by the diluting 500 μL standardised bacterial solution in 4500 μL PBS or sterile saline to obtain 107 CFU/mL.

### Microbiological Evaluation of Overnight Bacteria Suspensions

To model fluid exudate from a chronic wound, the wound dressing made according to ‘Modification of ActivHeal® Alginate’, above, was placed into an agar plate and the test pathogen was smeared over the surface of the agar.

The enzymatic substrates were tested in 4 different concentrations (300, 600, 900 and 3000 mg/L) using DI water as a solvent, and the solutions were absorbed into the wound dressing samples, which were dried overnight under vacuum at 40° C.

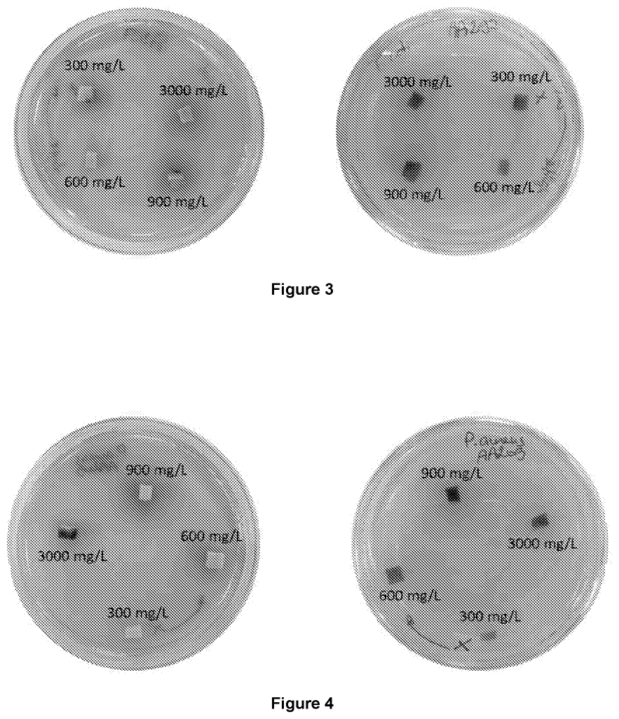

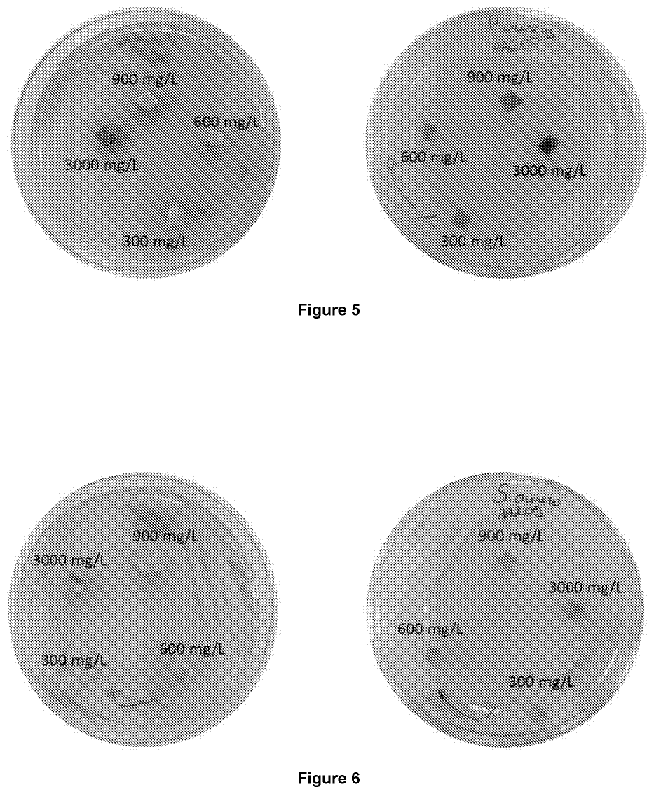

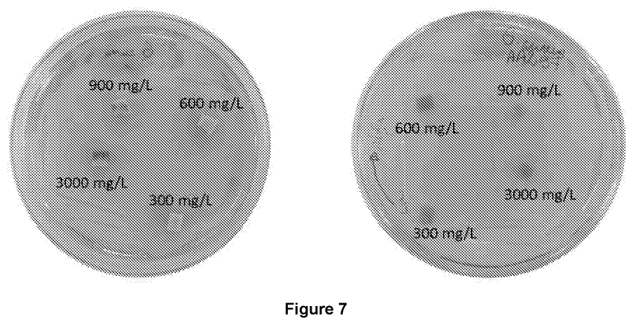

Two Gram negative strains (Escherichia coli—EC and Pseudomonas aeruginosa—PSA) and a Gram positive bacteria (Staphylococcus aureus—SA) were evaluated against the dimethyl (3a) and the diethyl (3b) substituted substrate. The overnight cultures were directly smeared from LB broth onto Columbia blood agar without measuring the OD. The wound dressings with the enzymatic substrate absorbed were placed onto the agar and were incubated overnight (see FIGS. 2-7).

### The Test Results are Summarised in

Table with the N,N-dimethyl substrate 3a and the N,N-diethyl substrate 3b. In Table 2, the intensities of the observable colour changes are: pale blue <light blue <blue <dark blue. The more intense the colour change, the easier it is to identify the presence of bacteria. The Gram-negative strains (EC and PSA) both produced blue colour changes with substrate 3b in 300 mg/L concentration, however SA only produced the colour change when higher (3000 mg/L) substrate 3b was applied. The N,N-diethyl substrate (3b) provided stronger colour changes than the dimethyl analogue (3a).

It is important to note that applying the substrate in very high concentrations (in this case 3000 mg/L) can result to the diffusion of the oxidative product of the hydrolysed aniline, which produced brown colouration of the agar.

TABLE 2
Microbiology evaluation of the overnight cultures against substrate
3a or 3b. Test results after 24 h incubation at 37° C.

Observable colour change

| 24 h | EC | PSA | SA |
|---|---|---|---|
| 3a (300 mg/L) | Light blue | None | None |
| 3a (600 mg/L) | Light blue | Light blue | Pale blue |
| 3a (900 mg/L) | Blue | Blue | None |
| 3a (3000 mg/L) | Dark blue | Blue | Light blue |
| 3b (300 mg/L) | Light blue | Light blue | None |
| 3b (600 mg/L) | Light blue | Light blue | None |
| 3b (900 mg/L) | Dark blue | Blue | None |
| 3b (3000 mg/L) | Dark blue | Dark blue | Blue |

### Microbiological Evaluation of Standardised Bacterial Suspensions

The microbiological evaluation of the overnight bacteria suspensions showed promising results. However, rapid evaluation of lower bacteria concentrations is essential for the desired clinical application. Three different solutions (300, 600 and 1500 mg/L) were prepared from the substrates 3a and 3b in micro filtered DI water as a solvent. The solutions were absorbed into the wound dressing samples, which were dried overnight under vacuum at 40° C.

Two Gram negative strains (Escherichia coli—EC and Pseudomonas aeruginosa—PSA) and a Gram-positive bacterium (Staphylococcus aureus—SA) were evaluated. The bacteria were incubated overnight, centrifuged at 3000×g for 5 mins, the supernatant was discarded, and the residue pellets were resuspended in 10 mL PBS. The inoculums were standardised by measuring the optical density at 600 nm to assure a 1-3×10⁸ CFU/mL. The standardised solutions were further diluted to prepare 1-3×10⁶ and 1-3×10⁴ CFU/mL suspension. 1×10⁶ CFU would be an ideal breakpoint for a positive result, as this concentration coincides well with a level of colonisation that would likely go on to produce an infection.

100 μL bacterial suspensions were smeared over agar plates. Wound dressing samples were added to the top of the agar and were rolled over a few times (to ensure a full coverage and to ease the visibility of the colour change). The plates were incubated at 37° C., and the results were recorded after 24 h, after 48 h, after 72 h. The results are shown in Table 3-5; − indicates no colour change, +− indicates relatively discernible blue colour change, + indicates a moderately strong blue colour change and ++ indicates a strong blue colour change.

TABLE 3
Microbiology evaluation of the standardised bacterial
solutions of EC, PSA, and SA with substrate 3a and
substrate 3b after 24 h incubation at 37° C.

| | Observable colour change | | | | | | | | |
|---|---|---|---|---|---|---|---|---|---|
| | EC | | | PSA | | | SA | | |
| 24 h | 10⁸ | 10⁶ | 10⁴ | 10⁸ | 10⁶ | 10⁴ | 10⁸ | 10⁶ | 10⁴ |
| 3a (300 mg/L) | +− | − | − | − | − | − | − | − | − |
| 3a (900 mg/L) | +− | − | − | − | − | − | − | − | − |
| 3a (1500 mg/L) | +− | +− | − | − | − | − | − | − | − |
| 3b (300 mg/L) | +− | − | − | − | − | − | − | − | − |
| 3b (900 mg/L) | +− | − | − | − | − | − | − | − | − |
| 3b (1500 mg/L) | +− | +− | − | − | − | − | − | − | − |

TABLE 4
Microbiology evaluation of the standardised bacterial
solutions of EC, PSA, and SA with substrate 3a and
substrate 3b after 48 h incubation at 37° C.

| | Observable colour change | | | | | | | | |
|---|---|---|---|---|---|---|---|---|---|
| | EC | | | PSA | | | SA | | |
| 48 h | 10⁸ | 10⁶ | 10⁴ | 10⁸ | 10⁶ | 10⁴ | 10⁸ | 10⁶ | 10⁴ |
| 3a (300 mg/L) | + | +− | − | − | − | − | − | − | − |
| 3a (900 mg/L) | + | +− | − | + | − | +− | − | − | − |
| 3a (1500 mg/L) | + | + | +− | + | +− | − | +− | − | − |
| 3b (300 mg/L) | + | + | + | + | − | − | − | − | − |
| 3b (900 mg/L) | ++ | + | + | ++ | +− | − | − | − | − |
| 3b (1500 mg/L) | ++ | + | + | ++ | − | − | +− | − | − |

TABLE 5
Microbiology evaluation of the standardised bacterial
solutions of EC, PSA, and SA with substrate 3a and
substrate 3b after 72 h incubation at 37° C.

| | Observable colour change | | | | | | | | |
|---|---|---|---|---|---|---|---|---|---|
| | EC | | | PSA | | | SA | | |
| 72 h | 10⁸ | 10⁶ | 10⁴ | 10⁸ | 10⁶ | 10⁴ | 10⁸ | 10⁶ | 10⁴ |
| 3a (300 mg/L) | ++ | + | − | + | + | + | − | − | − |
| 3a (900 mg/L) | ++ | + | + | ++ | ++ | ++ | − | − | − |
| 3a (1500 mg/L) | ++ | ++ | + | ++ | + | ++ | +− | − | − |
| 3b (300 mg/L) | + | + | + | + | + | + | − | − | − |
| 3b (900 mg/L) | ++ | ++ | ++ | ++ | ++ | ++ | − | − | − |
| 3b (1500 mg/L) | ++ | ++ | ++ | ++ | + | ++ | +− | +− | − |

After 24 h incubation EC (10⁸ CFU) showed a weak colour change with both substrates, however the blue colour was barely visible.

After 48 h incubation both Gram-negative strains, EC and PSA strains (10⁸ CFU) produced strong blue colour changes with substrate 3b at higher substrate concentration (900 or 1500 mg/L), while EC (10⁶ CFU and 10⁴ CFU) showed moderate colour change when lower bacterial concentrations were presented. Both Gram-negative bacteria, EC and PSA produced strong blue colour changes with the N,N-diethyl substrate 3b (900 mg/L) when higher or lower bacterial concentrations were presented (10⁸ CFU, 10⁶ CFU, and 104 CFU), while the N,N-dimethyl substrate 3a provided moderate or strong colour change at higher substrate concentrations (900 or 1500 mg/L). The evaluated Gram-positive organism, SA showed weak, but discernible colour change when the highest concentration of substrate evaluated (1500 mg/L of 3a or 3b) was present.

After 72 h both Gram-negative strains, EC and PSA, at 10⁸, 10⁶ or 10⁴ CFU produced moderate or strong blue colour changes with both substrates 3a and 3b at every tested substrate concentration. The evaluated Gram-positive organism, SA only showed weak, hardly visible colour change even when the highest concentration of substrate evaluated (1500 mg/L of 3a or 3b) was present.

1. A wound dressing comprising a modified polymer, wherein the modified polymer comprises a polymer backbone covalently coupled to a compound of Formula (I): 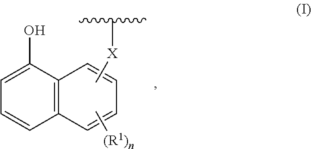
wherein X is a covalent bond or a linker;
R¹ is independently at each occurrence selected from halo, C₁₋₃ alkyl, C₂₋₃ alkenyl, C₁₋₃ haloalkyl, C₁₋₃ alkoxy, C₁₋₃ haloalkoxy and CN;
n is 0, 1, 2 or 3; and [symbol]
indicates the point of attachment to the polymer backbone.

2. The wound dressing of claim 1, wherein the linker comprises:
C₁₋₈ alkylene, —(OCH₂CH₂)ₘ—, —(CH₂CH₂O)ₘ—, —S—, —SO₂— or 1,2,3-triazolylene, wherein m is an integer selected from 1 to 20, and C₁₋₈ alkylene is optionally substituted with from 1 to 4 substituents selected from oxo, halo, C₁₋₃ alkoxy, C₁₋₃ haloalkoxy and CN; and/or
—Y—Z¹-L¹-Z²—, wherein Y is selected from —C(O)—, —S(O)₂— and —S(O)—; Z¹ and Z² are independently selected from O, S and NH; and L¹ is selected from C₁₋₈ alkylene, C₂₋₈ alkenylene and C₂₋₈ alkynylene, wherein L¹ is optionally substituted where chemically possible with from 1 to 4 substituents selected from oxo, halo, C₁₋₃ alkoxy, C₁₋₃ haloalkoxy and CN.

3. The wound dressing of claim 1, wherein the compound of Formula (I) is a compound of Formula (Ia): 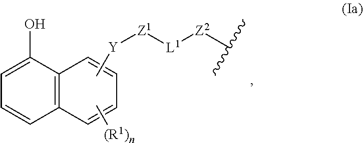
wherein Y is selected from —C(O)—, —S(O)₂— and —S(O)—;
Z¹ and Z² are independently selected from O, S and NH; and
L¹ is C₁₋₈ alkylene, C₂₋₈ alkenylene and C₂₋₈ alkynylene, wherein L¹ is optionally substituted where chemically possible with from 1 to 4 substituents selected from oxo, halo, C₁₋₃ alkoxy, C₁₋₃ haloalkoxy and CN.

4. The wound dressing of claim 1 wherein n is 0.

5. The wound dressing of claim 1 wherein the compound of Formula (I) is selected from:

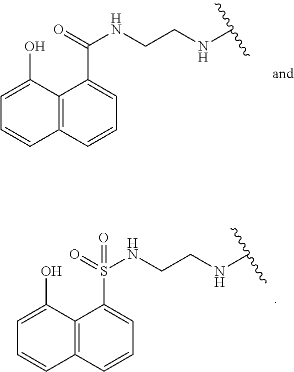 [SMILES: *NCCNC(=O)c1cccc2cccc(O)c12 ; *NCCNS(=O)(=O)c1cccc2cccc(O)c12]

6. The wound dressing of claim 5, wherein the polymer backbone comprises a —COOH or —COO⁻ group.

7. The wound dressing of claim 5, wherein the polymer backbone comprises a polysaccharide.

8. The wound dressing of claim 7, wherein the compound of Formula (I) is covalently coupled to the polysaccharide via an ester or an amide bond.

9. (canceled)

10. The wound dressing of claim 7, wherein the polysaccharide comprises alginic acid or a pharmaceutically acceptable salt thereof.

11. The wound dressing of claim 7, wherein the modified polymer comprises structural unit (A) and/or (B):

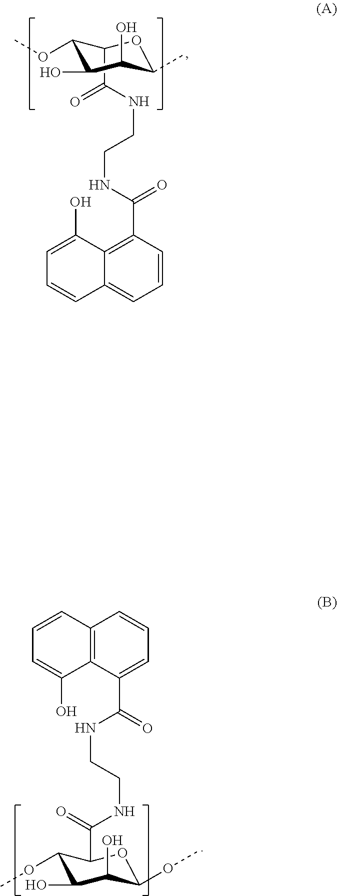

12. The wound dressing of claim 1, wherein the wound dressing further comprises an enzymatic substrate of Formula (II), 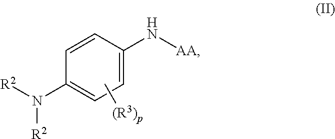
wherein AA is an amino acid bonded to the rest of the substrate via an amide bond;
R² is independently at each occurrence a C₁₋₅ alkyl;
p is 0, 1, 2 or 3; and
R³ is independently at each occurrence selected from halo, C₁₋₃ alkyl, C₂₋₃ alkenyl, C₁₋₃ haloalkyl, C₁₋₃ alkoxy, C₁₋₃ haloalkoxy and CN,
or a pharmaceutically acceptable salt thereof.

13. The wound dressing of claim 12, wherein the enzymatic substrate is a substrate of Formula (IIa): 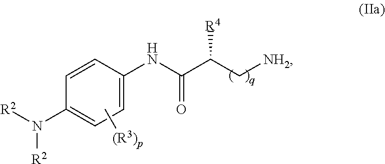
wherein R⁴ is selected from H and an amino acid side chain; and
q is 0 or 1,
or a pharmaceutically acceptable salt thereof.

14. (canceled)

15. The wound dressing of claim 12, wherein the substrate is selected from:

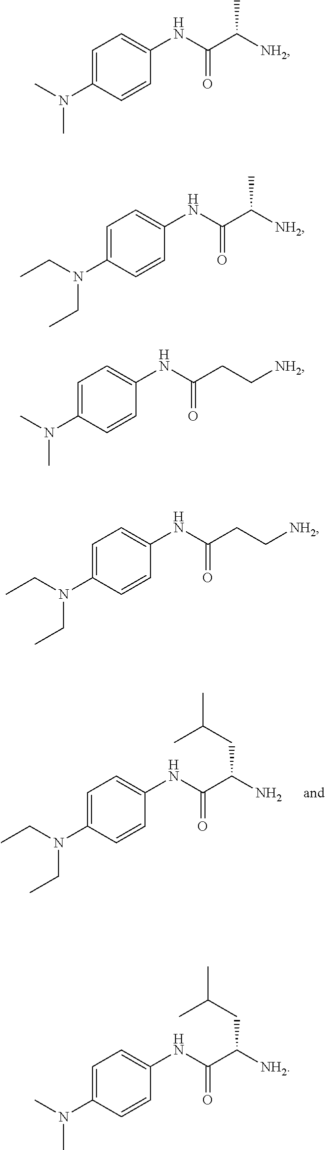 [SMILES: C[C@H](N)C(=O)Nc1ccc(N(C)C)cc1 ; CCN(CC)c1ccc(NC(=O)[C@H](C)N)cc1 ; CN(C)c1ccc(NC(=O)CCN)cc1 ; CCN(CC)c1ccc(NC(=O)CCN)cc1 ; CCN(CC)c1ccc(NC(=O)[C@@H](N)CC(C)C)cc1 ; CC(C)C[C@H](N)C(=O)Nc1ccc(N(C)C)cc1]

16. The wound dressing of claim 12, wherein the enzymatic substrate is absorbed into the wound dressing.

17. A sterile package comprising the wound dressing of claim 12.

18. (canceled)

19. A method for detecting the presence of bacteria in a wound, the method comprising applying the wound dressing of claim 12 to a wound, removing the wound dressing from the wound and observing any colouration on the wound dressing.

20. (canceled)

21. (canceled)

22. (canceled)

23. (canceled)

24. A method of forming a modified polysaccharide comprising a modified monosaccharide residue of Formula (III): 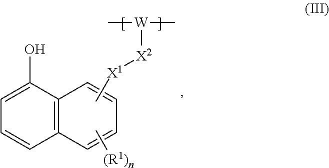
wherein X¹ is a covalent bond or a linker;
X² is selected from NH, O and S;
W is a monosaccharide residue;
R¹ is independently at each occurrence selected from halo, C₁₋₃ alkyl, C₂₋₃ alkenyl, C₁₋₃ haloalkyl, C₁₋₃ alkoxy, C₁₋₃ haloalkoxy and CN; and
n is 0, 1, 2 or 3,
the method comprising:
a) contacting an acidic polysaccharide with a compound of Formula (IV): 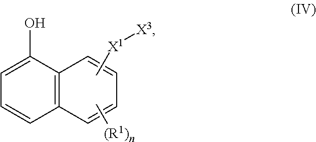
to form the modified polysaccharide,
wherein X³ is selected from —NH₂, —OH and —SH,
with the proviso that when X² is NH, X³ is —NH₂; when X² is O, X³ is —OH; and when X² is S, X³ is —SH.

25. (canceled)

26. (canceled)

27. The method of claim 24, wherein the method is a method of forming modified polysaccharide (C): 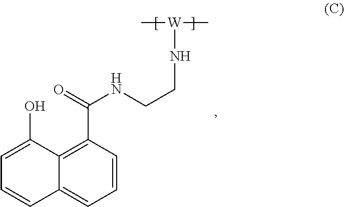
and the compound of Formula (IV) is compound (D): 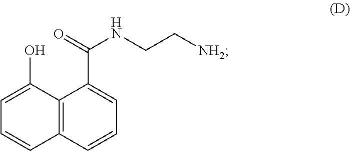 [SMILES: NCCNC(=O)c1cccc2cccc(O)c12]
or
wherein the method is a method of forming modified polysaccharide (E): 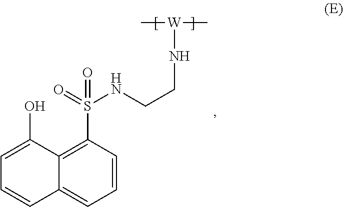
and the compound of Formula (IV) is compound (F):

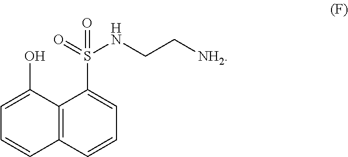 [SMILES: NCCNS(=O)(=O)c1cccc2cccc(O)c12]

28. (canceled)

29. (canceled)

30. A method of forming a wound dressing, wherein the wound dressing comprises a modified polysaccharide, wherein the method comprises forming the modified polysaccharide according to the method of claim 24 and optionally depositing an enzymatic substrate into the modified polysaccharide, wherein the enzymatic substrate is a substrate of Formula (11): 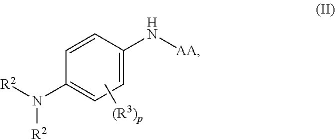
wherein AA is an amino acid bonded to the rest of the substrate via an amide bond:
R² is independently at each occurrence a C₁₋₅ alkyl:
p is 0, 1, 2 or 3; and
R³ is independently at each occurrence selected from halo, C₁₋₃ alkyl, C₂₋₃ alkenyl, C₁₋₃ haloalkyl, C₁₋₃ alkoxy, C₁₋₃ haloalkoxy and CN,
or a pharmaceutically acceptable salt thereof.

31. (canceled)

32. The method of claim 30, further comprising:
i) packaging the wound dressing to form a packaged wound dressing; and
ii) optionally sterilising the packaged wound dressing.
